(19) United States
(12) Patent Application Publication
(10) Pub. No.: US 2010/0196284 A1
(43) Pub. Date: Aug. 5, 2010

(54) Ultrasound Imaging with Targeted Microbubbles

(76) Inventors: Jonathan R. Lindner, Portland, OR (US); Beat Kaufmann, Oberwil (CH); Owen McCarty, Portland, OR (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/596,775

(22) PCT Filed: Apr. 18, 2008
(86) PCT No.: PCT/US08/60816

§ 371 Date: Mar. 29, 2010

Related U.S. Application Data
(60) Provisional application No. 60/913,086, filed on Apr. 20, 2007.
(60) Provisional application No. 60/947,844, filed on Jul. 3, 2007.

(51) Int. Cl. A61K 49/22; A61P 9/10

(52) U.S. Cl. 424/9.52; 424/9.1

(57) ABSTRACT

Compositions and methods for detecting various disorders with targeted microbubbles are disclosed. Specifically, microbubbles comprising glycoprotein Ib (GPIb), ligands for VCAM-1, and ligands for P-selectin such as PSGL-1 are disclosed. Also disclosed are methods for using targeted microbubbles to detect cardiovascular disease comprising administering the disclosed microbubbles to a subject and detecting the microbubbles in the vasculature using ultrasound.

This application claims priority under 35 U.S.C. §119(e) to U.S. Provisional Patent Application No. 60/913,086, filed on Apr. 20, 2007, and U.S. Provisional Patent Application No. 60/947,844, filed on Jul. 3, 2007. The foregoing applications are incorporated by reference herein.

Pursuant to 35 U.S.C. Section 202(c), it is acknowledged that the United States Government has certain rights in the invention described herein, which was made in part with funds from the National Institutes of Health/National Heart, Lung, and Blood Institute Grant Nos. R01-HL074443 and R01-HL078610.

### FIELD OF THE INVENTION

The present invention relates to the fields of imaging. Specifically, compositions and methods for detecting various disorders with targeted microbubbles are disclosed.

### BACKGROUND OF THE INVENTION

Several publications and patent documents are cited throughout the specification in order to describe the state of the art to which this invention pertains. Each of these citations is incorporated herein by reference as though set forth in full.

Ultrasound contrast agents have been developed in order to better define intracardiac contours and masses, to assess tissue perfusion, and to evaluate parenchymal masses (such as in the liver). These contrast agents are composed of air or gas filled microbubbles or nano-scale (<1 micron diameter) particles that are encapsulated with protein, lipid or bio-compatible polymers.

It has also been demonstrated that tissue inflammation can be assessed noninvasively by ultrasound imaging of microbubbles that are retained by activated leukocytes (Lindner et al. (2000) Circulation 102:531-538; Lindner et al. (2000) Circulation 102:2745-2750). Albumin and lipid microbubbles attach to leukocytes adherent to the venular endothelium and are phagocytosed intact within minutes (Lindner et al. (2000) Circulation 102:531-538; Lindner et al. (2000) Circulation 102:2745-2750; Lindner et al. (2000) Circulation 101:668-675). The ultrasound signal from these microbubbles, however, is relatively low because of the small proportion of microbubbles that are retained and viscoelastic damping of microbubbles once phagocytosed. This signal may be enhanced by incorporation of specific lipid moieties in the microbubble shell that enhance microbubble avidity for activated leukocytes (Lindner et al. (2000) Circulation 102:2745-2750).

A more direct method for assessing microvascular inflammatory responses is possible by conjugating ligands for specific endothelial cell adhesion molecules to the microbubble shell (Villanueva et al. (1998) Circulation 98:1-5). Potential advantages of this strategy include a greater number of retained microbubbles, less acoustic damping because the microbubbles remain extracellular, and the ability to quantify expression of specific adhesion molecules.

### SUMMARY OF THE INVENTION

In accordance with the present invention, methods for detecting cardiovascular diseases and disorders in a subject are provided. In a particular embodiment, the methods comprise administering to a subject microbubbles comprising a targeting ligand and monitoring the vascular retention of the microbubbles to determine the presence of the cardiovascular disease or disorder. Targeting ligands include, without limitation, GPIb, targeting ligands specific for P-selectin, and targeting ligands specific for VCAM-1. Cardiovascular diseases and disorders include, without limitation, atherosclerosis, ischemia, myocardial injury, ischemia-mediated angiogenesis, left ventricular ischemia, inflammation, thrombosis, and prothrombotic environment.

In accordance with another aspect of the instant invention, compositions comprising microbubbles comprising a targeting ligand and a carrier are provided. Targeting ligands include GPIb, targeting ligands specific for VCAM-1, and targeting ligands specific for P-selectin.

### BRIEF DESCRIPTIONS OF THE DRAWING

FIG. 1 is a graph depicting the attachment of control (MB_{c}) and P-selectin (MBₚ) targeted microbubbles (MB) as assessed by intravital microscopy on control and ischemic mice.

FIG. 2 is a graph depicting the retention of size segregated control (MB_{c}) and P-selectin (MBₚ) targeted microbubbles at the anterior and posterior myocardium.

FIG. 3 is a graph of the attachment of microbubbles comprising rPSGL-IG (MB_{PSGL}) and microbubbles comprising antibodies to P-selectin (MB_{Ab}) to P-selectin labeled flow chambers at increasing shear stress levels.

FIG. 4A is a graph of the venular endothelial attachment of MB_{PSGL} and MB_{Ab} as assessed by intravital microscopy. FIG. 4B is a graph of the number of MB_{PSGL} and MB_{Ab} in a given optical field.

FIG. 5 provides pseudocolorized images from intravital microscopy illustrating microbubble adherence to small venules. Images were generated by superimposition of individual images with separate fluorescent filters for DiI-labeled MB_{Ab} (red) and DiO-labeled MB_{PSGL} (green).

FIG. 6 is a graph of the mean signal intensity of microbubbles comprising a control antibody (MB_{C}), MB_{PSGL}, and MB_{Ab} in the control leg and ischemic leg of wild-type and P-selectin^{-/-} mice and control mice without ischemia.

FIG. 7 provides illustrative images from targeted contrast-enhanced ultrasound with MB_{C}, MB_{PSGL}, and MB_{Ab} in the wild-type and P-selectin^{-/-} mice.

FIG. 8A is a graph of the mean (±SEM) number of control (MB_{C}) and VCAM-1-targeted (MB_{V}) microbubbles attached to non-stimulated and TNF-α-stimulated SVECs at a shear rate of 0.5 dyne/cm². *p<0.01 vs MB_{C}; †tp=0.05 vs −TNF-α. FIG. 8B is a graph of the attachment of VCAM-1-targeted microbubbles to TNF-α-stimulated SVECs at variable shear rates. Because shear was varied by flow rate, data are expressed as percentage of total number transiting through the entire chamber. FIG. 8C is a graph of the VCAM-1-targeted microbubble attachment at high shear rates of 8 or 12 dyne/cm² after 5 minutes of continuous flow (baseline, BL) and after sequential brief pauses (Pₙ) where shear was reduced to <0.5 dyne/cm². ANOVA values represent the trend towards increased attachment with sequential pauses. FIG. 8D presents images of examples of a single optical field under light and fluorescent microscopy images demonstrating DiI-labeled VCAM-1-targeted microbubble attachment to SVECs. Scale bar=20 μm.

FIG. 9A is a graph of microbubble attachment to the thoracic aorta 10 minutes after intravenous injection assessed by ex vivo fluorescent microscopy. Mean (±SEM) attachment of control (MB_{c}) and VCAM-1-targeted (MB_{V}) microbubbles is presented. *p<0.05 vs. MB_{C}; †tp=0.05 vs. MB_{V} in wild-type on chow diet; ‡tp<0.05 vs. MB_{V} in all other groups. FIG. 9B provides examples of en face dual-fluorescent microscopy of the thoracic aorta. On fluorescent epi-illumination, DiI-labeled VCAM-1-targeted microbubbles appear red (observed) while DiO-labeled control microbubbles appear green (not observed). Examples of the ApoE^{-/-} mouse on chow diet are shown for regions with and without evidence for irregular wall thickening on transillumination. Scale bar=25 μm.

FIGS. 10A-10H are images of the distribution of non-targeted microbubbles in transit through the aortic lumen assessed by high-frequency (30 MHz) contrast-enhanced ultrasound (CEU) acquired at a frame rate of 20 Hz. FIG. 10A provides illustrations of regions-of-interest spanning from position 1 (adjacent to the greater curvature) to position 5 (adjacent to the lesser curvature). FIGS. 10B to 10C are images of the maximum intensity projections taken 400 ms apart as microbubbles appear in the aorta, thereby demonstrating diffuse distribution of microbubbles throughout the lumen. FIG. 11i provides a graph which depicts CEU maximum intensity projection data for the different regions-of-interest.

FIGS. 11A-11D provide representative images from an ApoE^{-/-} mouse on a hypercholesterolemic diet (HCD). FIG. 11A is an image of the aortic arch by 2-D ultrasound imaging (Ao); FIG. 11B is an image of the pulsed-wave Doppler imaging of the arch; and FIGS. 11C and 11D are contrast-enhanced ultrasound images of the aortic arch 10 minutes after intravenous injection of either VCAM-1-targeted microbubbles (FIG. 110) or control microbubbles (FIG. 11D). Color scale for the contrast ultrasound images is at the bottom of each frame and each targeted imaging example is shown after correction for signal from freely-circulating microbubbles.

FIGS. 12A and 12B are graphs of the non-attenuated peak negative acoustic pressure measurements at the focal depth for the linear-array transducer used for targeted CEU imaging. FIG. 12A is a graph of the peak negative acoustic pressure according to in-plane lateral position and elevational position. FIG. 12B is a graph of the elevational dimension power profile averaged from all lateral positions. The average cross-sectional internal dimension of the aorta (1.3 mm) is superimposed on the elevation plane power profile.

FIG. 13 is a graph of the background-subtracted CEU signal intensity from the aortic arch 10 minutes after intravenous injection of control (MB_{C}) and VCAM-1-targeted (MB_{V}) microbubbles in the different animal groups. Data depict median value (horizontal line), 25-75% percentiles (box), and range of values (whiskers). *p<0.05 versus MB_{V} in wild-type mice on chow diet. †tp<0.001 versus MB_{V} in other animal groups.

FIGS. 14A-14F are representative images of VCAM-1 staining by immunohistochemistry of the thoracic aorta. FIG. 14 A is an image from a wild-type mouse on chow diet demonstrating minimal endothelial VCAM-1 staining. FIG. 14B is an image from wild type mouse on HCD demonstrating VCAM-1 expression localized to the luminal endothelial surface. FIGS. 14C and 14D are images from an Apo E^{-/-} mouse on chow diet demonstrating VCAM-1 staining particularly on the endothelial surface overlying regions of neointimal thickening. FIGS. 14E and 14F are images from an Apo E^{-/-} mouse on HCD demonstrating robust VCAM-1 staining throughout the aorta but especially on the endothelial surface overlying severe plaque formation and on cells within the neointima.

FIG. 15 is a graph of the attachment of microbubbles comprising BSA (MB_{BSA}) or GPIb (MB_{GPIb}) to immobilized VWF under a shear of 2 dyn/cm².

FIG. 16A provides a contrast image of a collagen-coated string within the left ventricle of a rat. This is a baseline image, confirming the assumed clot location. The imaging power is 10 MHz, with a 19 Hz frame rate. FIG. 16B provides a contrast enhance ultrasound image of targeted, GPIbα-conjugated microbubbles attached to the clot. The imaging power is 7 MHz with an 18 Hz frame rate and a mechanical index of 0.14.

### DETAILED DESCRIPTION OF THE INVENTION

In accordance with the present invention, microbubble compositions targeted to bind to specific substrates are provided. Methods for the detection, diagnosis, and prognosis of various disorders using the microbubbles of the instant invention are also provided.

### I. DEFINITIONS

The following definitions are provided to facilitate an understanding of the present invention:

The term “functional” as used herein implies that the nucleic or amino acid sequence is functional for the recited assay or purpose.

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g. chromatographic methods, agarose or polyacrylamide gel electrophoresis, HPLC analysis, and the like).

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

An “antibody” or “antibody molecule” is any immunoglobulin, including antibodies and fragments thereof, that binds to a specific antigen. The term includes polyclonal, monoclonal, chimeric, single domain (Dab) and bispecific antibodies. As used herein, antibody or antibody molecule contemplates recombinantly generated intact immunoglobulin molecules and immunologically active portions of an immunoglobulin molecule such as, without limitation: Fab, Fab′, F(ab′)₂, Fv, scFv, scFv₂, scFv-Fc, minibody, diabody, tetrabody, single variable domain (e.g., variable heavy domain, variable light domain), bispecific, Affibody® molecules (Affibody, Bromma, Sweden), and peptabodies (Terskikh et al. (1997) PNAS 94:1663-1668). Methods for recombinantly producing antibodies are well-known in the art.

With respect to antibodies, the term “immunologically specific” refers to antibodies that bind to one or more epitopes of a protein or compound of interest, but which do not substantially recognize and bind other molecules in a sample containing a mixed population of antigenic biological molecules.

The term “conjugated” or “linked” may refer to the joining by covalent or noncovalent means of two compounds or agents of the invention.

As used herein, “diagnosis” refers to providing any type of diagnostic information, including, but not limited to, whether a subject is likely to have a condition, information related to the nature or classification of the condition, information related to prognosis and/or information useful in selecting an appropriate treatment. As used herein, “diagnostic information” or information for use in diagnosis is any information that is useful in determining whether a patient has a disease or condition and/or in classifying the disease or condition into a phenotypic category or any category having significance with regards to the prognosis of or likely response to treatment (either treatment in general or any particular treatment) of the disease or condition.

As used herein, “ischemia” is a reduction in blood flow. Ischemia can be caused by the obstruction of an artery or vein by a blood clot (thrombus) or by any foreign circulating matter (embolus), or by a vascular disorder such as atherosclerosis. Reduction in blood flow can have a sudden onset and short duration (acute ischemia) or can have a slow onset with long duration or frequent recurrence (chronic ischemia).

As used herein, “thrombus” refers to any semi-solid aggregate of blood cells enmeshed in fibrin and clumps of platelets originating from platelets actively binding to the solid-phase agent. Thrombosis refers to the formation of a thrombus within a blood vessel. A prothrombotic environment refers to an increased tendency towards thrombosis.

Generally, cardiovascular diseases or disorders refer to the class of diseases or disorders that involve the heart and/or blood vessels.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), bulking substance (e.g., lactose, mannitol), excipient, auxilliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, 20th Edition, (Lippincott, Williams and Wilkins), 2000; Liberman, et al., Eds., Pharmaceutical Dosage Forms, Marcel Decker, New York, N.Y., 1980; and Kibbe, et al., Eds., Handbook of Pharmaceutical Excipients (3.sup.rd Ed.), American Pharmaceutical Association, Washington, 1999.

### II. MICROBUBBLES

In general, microbubbles are gas bubbles having a diameter of a few microns (e.g., about 1-10 μm, particularly about 1-5 μm) dispersed in an aqueous medium. The microbubbles may be spherical or non-spherical. The sphericity of the microbubbles can be altered, for example, by manipulating the shape of the envelope or shell encompassing the gas or by generating folds, projections, wrinkles, or the like in the membrane (see, e.g., U.S. Patent Application Publication No. 2005/0260189).

Typically, microbubbles are in aqueous suspensions in which the microbubbles of gas or air are bounded at the gas/liquid interface by a very thin envelope of surfactants (amphiphilic material) disposed at the gas to liquid interface. Microbubbles may also be bubbles of gas that are surrounded by a solid material envelope formed of natural or synthetic polymers (see, e.g., European patent application EP 0458745). However, microbubbles comprising an envelope of an amphiphilic material are preferred.

Formulations for microbubbles are known in the art. For example, microbubble suspensions may be prepared by contacting powdered amphiphilic materials (e.g. freeze-dried preformed liposomes or freeze-dried or spray-dried phospholipid suspensions) with air or other gas and then with aqueous carrier and then agitating to generate a microbubble suspension. Examples of aqueous suspensions of gas microbubbles and preparation thereof can be found for instance in U.S. Pat. Nos. 5,271,928; 5,445,813; 5,413,774; 5,556,610; 5,597,549; and 5,827,504; WO 97/29783; WO 94/01140; and U.S. Patent Application Publication Nos. 2006/0034770; 2003/0017109; 2004/0126321; 2005/0207980; and 2005/0260189.

The gas of the microbubble may comprise, without limitation, at least one of: air, nitrogen, oxygen, carbon dioxide, hydrogen, an inert gas (e.g., helium, argon, xenon or krypton), a sulphur fluoride (e.g., sulphur hexafluoride, disulphur decafluoride or trifluoromethylsulphur pentafluoride), selenium hexafluoride, an optionally halogenated silane such as methylsilane or dimethylsilane, a low molecular weight hydrocarbon (e.g., containing up to 7 carbon atoms; including, without limitation, alkanes (e.g., methane, ethane, propane, butane or pentane), cycloalkanes (e.g., cyclopropane, cyclobutane or cyclopentane), alkenes (e.g., ethylene, propene, propadiene or a butane), or alkynes (e.g., acetylene or propyne)), an ether (e.g., dimethyl ether), a ketone, an ester, and a halogenated (preferably fluorinated) low molecular weight hydrocarbon (see, generally, U.S. Pat. No. 6,264,917). In a particular embodiment, the interior of the microbubbles may exclude liquids.

Microbubbles may be targeted to specific molecules or target cells or tissues by affixing at least one targeting molecule to the outer surface of the bubble. This allows spatially localized detection of pathology in a tissue under investigation, in addition to the possibility of delivering bioactive substances to said tissue. Methods of generating microbubbles with desired targeting ligands are also known in the art. Targeting ligands may be linked or coupled to the microbubbles by any method. Exemplary methods are provided in U.S. Pat. Nos. 6,264,917; 6,245,318; 6,331,289; and 6,443,898.

In a particular embodiment, the targeting ligands are coupled to the microbubbles via a biotin-avidin-biotin bridge. For example, the microbubbles and targeting ligands may be biotinylated and a biotin binding agent (e.g., streptavidin) may be used to bind both the biotinylated targeting ligand and the biotinylated microbubble. As used herein, “biotin binding agent” encompasses, without limitation, avidin, streptavidin and other avidin analogs such as streptavidin or avidin conjugates, highly purified and fractionated species of avidin or streptavidin, and non or partial amino acid variants, recombinant or chemically synthesized avidin analogs with amino acid or chemical substitutions which still accommodate biotin binding. Preferably, each biotin binding agent molecule binds at least two biotin moieties and more preferably at least four biotin moieties. Additionally, as used herein, “biotin” encompasses biotin in addition to biocytin and other biotin analogs such as biotin amido caproate N-hydroxysuccinimide ester, biotin 4-amidobenzoic acid, biotinamide caproyl hydrazide and other biotin derivatives and conjugates. Other derivatives include biotin-dextran, biotin-disulfide-N-hydroxysuccinimide ester, biotin-6 amido quinoline, biotin hydrazide, d-biotin-N-hydroxysuccinimide ester, biotin maleimide, d-biotin p-nitrophenyl ester, biotinylated nucleotides and biotinylated amino acids such as N-biotinyl-1-lysine.

Any compound that aids in the formation and maintenance of the bubble membrane or shell by forming a layer at the interface between the gas and liquid phases may be used. The microbubbles of the instant invention may comprise one or more different types of surfactants. Surfactants include, without limitation, lipids, sterols, hydrocarbons, fatty acids, amines, esters, sphingolipids, thiol-lipids, phospholipids, nonionic surfactants, neutral or anionic surfactants, and derivatives thereof. The surfactants may be natural or synthetic. U.S. Patent Application Publication No. 2005/0260189 provides examples of surfactants that may be employed in the synthesis of microbubbles.

Microbubbles of the instant invention may also comprise at least one detectable label. In a particular embodiment, the detectable label is a fluorescent label such as dialkylcarbocyanine probes (e.g., DiI and DiO).

While microbubbles are exemplified throughout the instant application, nanobubbles (diameter about 5 to 900 nm) may also be used.

### III. MICROBUBBLE TARGETING LIGANDS

The microbubbles of the instant invention may comprise at least one targeting ligand. Preferred targets and targeting ligands of the instant invention are set forth below.

### A. P-Selectin

In a particular embodiment, the microbubbles of the instant invention comprise targeting ligands directed to P-selectin. P-selectin is an endothelial cell adhesion molecule expressed during inflammatory responses (Bevilacqua et al. (1993) J. Clin. Invest. 91:379-387) and ischemia-reperfusion (Kanwar et al. (1998) Microcirculation 5:281-287). P-selectin participates in the capture of leukocytes and rolling in venules. Lipid microbubbles bearing antibodies to P-selectin provide a means to image early inflammatory responses when intravenously administered (Lindner et al. (2001) Circulation 104:2107-2112). More specifically, the microbubbles were tested in wild-type and P-selectin-deficient (P^{-/-}) mice with intravital microscopy and by performing contrast-enhanced renal ultrasound early after ischemia-reperfusion injury.

In a preferred embodiment, the targeting ligand is a fusion protein comprising a P-selectin ligand and a dimerization domain. The P-selectin ligand may be a soluble P-selectin ligand protein or fragment thereof having P-selectin binding activity. In a particular embodiment, the ligand is P-selectin glycoprotein ligand-1 (PSGL-1) or a fragment thereof capable of binding P-selectin. U.S. Patent Application Publication No. 2003/0166521 provides examples of P-selectin ligands and fragments thereof.

As used herein, the term “dimerization domain” refers to a protein binding domain (of either immunological or non-immunological origin) that has the ability to bind to another protein binding domain with sufficient strength and specificity such as to form a dimer. Examples of dimerization domains include, without limitation, an Fc region, a hinge region, a CH3 domain, a CH4 domain, a CH1-CL pair, a leucine zipper (e.g. a jun/fos leucine zipper (Kostelney et al., J. Immunol. (1992) 148:1547-1553) or a yeast GCN4 leucine zipper), an isoleucine zipper, a receptor dimer pair (e.g., interleukin-8 receptor (IL-8R) and integrin heterodimers such as LFA-1 and GPIIIb/IIIa) or the dimerization region(s) thereof, dimeric ligand polypeptides (e.g., nerve growth factor (NGF), neurotrophin-3 (NT-3), interleukin-8 (IL-8), vascular endothelial growth factor (VEGF), VEGF-C, VEGF-D, PDGF members, and brain-derived neurotrophic factor (BDNF) (Arakawa et al. (1994) J. Biol. Chem. 269:27833-27839; Radziejewski et al. (1993) Biochem. 32:1350) or the dimerization region(s) thereof, a pair of cysteine residues able to form a disulfide bond, a pair of peptides or polypeptides, each comprising at least one cysteine residue (e.g., from about one, two or three to about ten cysteine residues) such that disulfide bond(s) can form between the peptides or polypeptides, and antibody variable domains. In a preferred embodiment, the dimerization domain is an Fc domain of an immunoglobulin. The dimerization domain and the P-selectin antagonist may be linked directly to each other (e.g., covalently attached) or may be connected via a linker domain. U.S. Patent Application Publication No. 2003/0166521 provides examples of fusion proteins comprising P-selectin ligands and the Fc domain of immunoglobulin.

Ischemia, such as myocardial ischemia, can be detected by molecular imaging of inflammation with myocardial contrast echocardiography and microbubbles targeted to the adhesion molecule P-selectin.

### B. VCAM-1

The critical role that inflammation plays in atherosclerosis has produced significant interest in better methods to evaluate it. Ideally such techniques should 1) be specific for inflammatory responses that occur in the vasculature, 2) be sufficiently sensitive to detect early events, 3) be able to provide spatial information, and 4) be practical in terms of cost, speed and ease of use in order to be used as a rapid screening tool. To that end, it was investigated whether CEU molecular imaging could be used to evaluate expression of the endothelial cell adhesion molecule VCAM-1 in murine models of atherosclerosis. VCAM-1-targeted signal enhancement in the different animal groups in this study varied according to the severity of atherosclerotic plaque development.

A method for imaging vascular inflammation may have a major impact in both the clinical and research laboratory settings. Strategies that are used currently to evaluate risk of cardiovascular disease or major adverse cardiac events may not necessarily meet the clinical needs of the future given the trend towards earlier and more aggressive therapy. The Framingham risk score and modifications thereof take into account multiple different clinical variables. However, about 40% of the adult U.S. population falls into an intermediate risk category (Jacobson et al. (2000) Arch. Intern. Med., 160:1361-9) with a 6 to 20% risk of developing symptomatic coronary heart disease within the ensuing 10 years. Further refinement in risk stratification for this intermediate risk category is desirable in order to make better use of long-term preventive therapies. There is also the notion that atherosclerosis, like many other diseases, is most amenable to treatment at an early stage. Efforts are underway to create novel therapies aimed at interrupting the inflammatory events that initiate plaque formation and trigger secondary growth responses. If treatment is to be initiated years to decades before atherosclerosis would otherwise become clinically evident, then a method for accurately detecting vascular inflammation would seem a critical factor.

Methods currently used to evaluate those who have developed symptoms of cardiovascular disease are designed to measure either the anatomic severity of disease or the physiologic consequences of increased circuit resistance, such as ischemia or reduced flow reserve. Imaging the inflammatory phenotype in those patients will likely add unique information, since inflammation is a key factor in the progression to unstable disease. The recruitment of inflammatory cells to the neointima results in release of prothrombotic, pro-mitogenic, pro-angiogenic, and detrimental vasoactive molecules; release of oxygen-derived free radicals; and production of proteases that contribute to adverse remodeling and erosion of the plaque protective barrier. It is necessary that new methods for evaluating inflammation should occur in parallel with new therapeutic strategies. Likewise, the use of molecular imaging in the pre-clinical development of therapies would provide a means to assess the pathogenic pathways being targeted. For this application, a technique should be quantitative, have high-throughput capacity, and possess sufficiently high-resolution for small animal model testing.

Molecular imaging with CEU has great promise for evaluating the inflammatory phenotype in atherosclerosis in patients due to the practical considerations mentioned herein and the balance between high sensitivity for tracer detection and spatial resolution. As described hereinbelow, microbubble contrast agents were targeted to VCAM-1. Microbubble contrast agents are pure intravascular agents and, accordingly, do not have access to extravascular events or epitopes that have been proposed for targeting such as resident inflammatory cells (macrophages, T-lymphocytes), proteases, or oxidation byproducts (Schafers et al. (2004) Circulation 109:2554-9; Deguchi et al. (2006) Circulation 114:55-62; Tsimikas et al. (1999) J. Nucl. Cardiol., 6:41-53; Ruehm et al. (2001) Circulation 103:415-22). Instead, an endothelial cell adhesion molecule that is a critical participant in inflammatory cell recruitment in atherosclerosois was targeted. VCAM-1 is present on endothelial cells early during the development of atherosclerosis and is otherwise expressed only in very low levels (Nakashima et al. (1998) Arterioscler. Thromb. Vasc. Biol., 18:842-511; Iiyama et al. (1999) Circ. Res., 85:199-207). VCAM-1 has been investigated as a potential target for molecular imaging in mice with other imaging techniques such as targeted infra-red and magnetic resonance probes (Kelly et al. (2005) Circ. Res., 96:327-36; Nahrendorf et al. (2006) Circulation 114:1504-11). In these studies, VCAM-1 signal in advanced stages of disease decreased with statin therapy, suggesting that the effects of therapy could be monitored with molecular imaging (Nahrendorf et al. (2006) Circulation 114:1504-11). Information from microbubble targeting is different from these diffusible tracers in that only endothelial VCAM-1 expression will be detected.

For targeting purposes, monoclonal antibodies against the extracellular domain of VCAM-1 were conjugated to the surface of the microbubbles, as described hereinbelow. This construct is characterized by an average of over 50,000 antibodies per microbubble and a surface density of several thousand per μm². One concern with such targeting is that, in the mouse aorta, peak wall shear stress can reach up to 80 to 90 dynes/cm², (Eriksson et al. (2000) Circ. Res., 86:526-33; Greve et al. (2006) Am. J. Physiol. Heart Circ. Physiol., 291:H1700-H1708) and the pulsatile variations in flow and thus wall shear stress is high. Despite this problem, there has been successful targeting of smaller echogenic liposomes to vascular surface epitopes in large animal models of atherosclerosis (Hamilton et al. (2004) J. Am. Coll. Cardiol., 43:453-60; Demos et al. (1999) J. Am. Coll. Cardiol., 33:867-75). These studies demonstrated conclusively that endothelial cell adhesion molecules could be targeted with acoustically active compounds. Furthermore, as shown hereinbelow, flow chamber experiments demonstrated that VCAM-1-targeted microbubble attachment efficiency was low during continuous high shear. However, a marked increase in attachment occurred when very high shear was interrupted briefly. Resumption of flow at high shear stress did not dislodge these microbubbles even at the maximum shear rate (12 dynes/cm²) that could be withstood without detachment of the SVECs from fibronectin-coated plates. Flow chamber experiments with precipitated Fc-VCAM-1 chimera have demonstrated the ability of VCAM-1-targeted microbubbles to firmly adhere even at shear rates of 50 and 90 dynes/cm². However, the en face microscopy studies described hereinbelow of the aortic arch 10 minutes after intravenous injection of fluorescent microbubbles provide evidence that microbubbles can attach in high-density to the aortic arch in vivo despite high peak shear stresses during systole.

Molecular imaging of VCAM-1 has the potential to diagnose inflammatory processes that initiate atherosclerosis long before symptoms arise. The data presented hereinbelow showing VCAM-1-targeted microbubble attachment and signal enhancement in wild-type mice on hypercholesterolemic diet (HCD) without evidence of plaque development indicate that early inflammatory changes can be detected. The finding that targeted microbubble attachment and signal enhancement was much greater in ApoE^{-/-} mice on HCD indicates that varying degrees of inflammatory response can be discerned. These mice not only had the greatest extent of endothelial VCAM-1 expression, but also the most severe form of disease in terms of plaque burden and the number of VCAM-1-expressing cells (macrophages) within the plaque. In these mice, both CEU and en face microscopy were consistent with a diffuse and widespread attachment of VCAM-1-targeted microbubbles, the density of which was within the dynamic range for detection of microbubbles attached to a 2-D surface (Lankford et al. (2006) Invest. Radiol., 41:721-8). The diffuse nature of attachment suggests that a surrogate large vessel may be used for evaluation when vascular inflammatory status is severe, although this was not directly tested. In ApoE^{-/-} mice on chow diet, attachment of microbubbles targeted to VCAM-1 was more pronounced in regions of atherosclerotic plaque, consistent with reports on upregulation of VCAM-1 predominantly in regions prone to plaque development (Nakashima et al. (1998) Arterioscler. Thromb. Vasc. Biol., 18:842-51). In the control wild type mice on chow diet, attachment of VCAM-1 targeted microbubbles was not different from control microbubbles, reflecting low or absent expression of VCAM-1 (Nakashima et al. (1998) Arterioscler. Thromb. Vasc. Biol., 18:842-51; Iiyama et al. (1999) Circ. Res., 85:199-207). This latter finding is important when considering the need for disease specificity (low false positive rate) required for a screening test.

The results of the studies presented hereinbelow indicate that contrast ultrasound with targeted microbubbles can detect inflammatory processes in atherosclerosis and discriminate the severity of inflammatory burden. Consequently, molecular imaging using targeted microbubbles and ultrasound can be used in the early diagnosis of atherosclerosis and in monitoring the efficacy of therapeutic interventions.

### C. GPIb

Treatment of diseases such as stroke, myocardial infarction and deep vein thrombosis rely on early diagnosis and the ability to locate vascular clots. Currently, reliable methods for the detection and localization of i) atrial appendage clots and ii) carotid thrombi are limited. This is particularly important in the elderly population where the therapeutic intervention (anticoagulation) must be weighed against the risks involved (bleeding diatheses). The detection of left atrial thrombus formation that occurs in approximately 15% of patients with atrial fibrillation (prevalence >2% of U.S. population over the age of 60) requires invasive transesophageal imaging because of the relatively low sensitivity of non-invasive transthoracic imaging. Moreover, there is no current method for evaluating microvascular thrombus formation that plays an important role in the pathophysiology of myocardial infarction and stroke. vWF/thrombin-targeted microbubbles will serve as novel CEU agents to facilitate the identification and localization of vascular clots. Furthermore, thrombus-bound microbubbles may have therapeutic potential as ultrasound-mediated sonolytic agents (“clot-busting” phenomenon), or releasing clot dissolving agents such as tissue plasminogen activator (TPA) (Corti et al. (2002) Am. J. Med., 113:668-680). An imaging technique that is simultaneously capable of non-invasively detecting and dissolving vascular clots would be invaluable in patients suffering from stroke or myocardial infarction.

In a particular embodiment, microbubbles comprising a targeting ligand to von Willebrand factor (VWF) can be used to diagnose thrombotic thrombocytopenic purpura (TTP). TTP is a life-threatening, multisystemic disorder resulting from the formation of platelet microthrombi (Moake, J. L. (2004) Semin. Hematol., 41:4-14; Moake, J. L. (2007) J. Clin. Apher., 22:37-49; Sadler et al. (2004) Hematology Am. Soc. Hematol. Educ. Program., 407-423; Moake, J. L. (2002) N. Engl. J. Med., 347:589-600; Moake, J. L. (2002) Annu. Rev. Med., 53:75-88), which in turn results from the incomplete processing of the adhesive protein VWF. In TTP, VWF-induced platelet aggregates form in the microcirculation throughout the body, causing partial occlusion of vessels and leading to organ ischemia, thrombocytopenia, and erythrocyte fragmentation. Presently, the TTP mortality rate is about 95% for untreated cases. In contrast, the survival rate is 80-90% with early diagnosis and treatment with plasma infusion and plasma exchange. However, at present, the detection of TTP relies on clinical diagnosis of a pentad of signs and symptoms, as there is no pathognomonic laboratory assay for TTP. Thus, the contrast-enhanced ultrasound (CEU) molecular imaging methods of the instant invention with microbubbles that target VWF (e.g., microbubbles comprising via the high-affinity platelet receptor glycoprotein (GP) Ib) may be used to diagnose and/or detect TTP.

For most cases of both familial and acquired idiopathic TTP, the underlying defect is due to endothelial cell (EC) secretion and release of ultralarge (UL) multimers of the adhesive protein VWF. Under normal conditions, monomers of VWF (280 kD) are linked by disulfide bonds to form UL multimers with various molecular masses that range into the millions of Daltons. The majority of UL multimers of VWF are constructed within ECs and stored in Weibel-Palade bodies (Ruggeri, Z. M. (2003) J. Thromb. Haemost., 1:1335-1342). These EC-produced ULVWF multimers are much larger than those found circulating in normal plasma, and they bind more efficiently to the platelet GPIb receptors for VWF than do the largest plasma VWF multimers. ECM-bound VWF plays a critical role in the tethering of platelets at high shear levels due to the unique, rapid on-rate of binding between VWF and the platelet receptor GPIb (Andre et al. (2000) Blood 96:3322-3328; Andrews et al. (2004) Thromb. Res., 114:447-453). The rapid on-rate of GPIb-VWF binding assists the recruitment of platelets to surface-bound VWF in the presence of shear forces produced by blood flow (Ruggeri, Z. M. (2002) Nat. Med., 8:1227-1234). The initial attachment of only a small quantity of ULVWF to the high-affinity platelet GPIb receptor is sufficient to mediate platelet recruitment and aggregation, resulting in rampant pathological microthrombi formation.

Under normal physiological conditions, the VWF-cleaving metalloprotease ADAMTS-13 prevents the entrance of ULVWF multimers in the circulation (Levy et al. (2005) Blood 106:11-17). ADAMTS-13 degrades the ULVWF multimers directly on the EC surface by cleaving peptide bonds in monomeric subunits of VWF, at position 842-843. However, ADAMTS-13 activity is undetectable or barely detectable due to the production of ADAMTS-13 autoantibodies in acquired idiopathic TTP or by ADAMTS-13 gene mutations in familial TTP. In the absence of ADAMTS-13, the ULVWF multimers are not cleaved upon secretion from ECs; instead, they remain anchored to the ECs, in long strings. Passing platelets adhere to these long ULVWF multimers via GPIb receptors, but do not adhere to the smaller VWF forms produced by cleavage of ULVWF under normal conditions (Bernardo et al. (2005) J. Thromb. Haemost., 3:562-570). Therefore, the presence of ULVWF multimers on the EC surface due to an insufficiency in ADAMTS-13 represents a key component in TTP pathogenesis.

There is currently great interest in cardiology and neurology in the ability to detect “vulnerable” atherosclerotic lesions that identify a patient at high risk for adverse cardiac or neurologic complications. Plaque rupture and subsequent vascular thrombus formation is the most common inciting factor in ischemic cardiovascular events. The ability to detect prothrombotic endothelial phenotype will be useful for early identification of high risk individuals and for selecting optimal treatment strategies. Moreover, abnormal endothelial expression of adhesion molecules such as vWF will provide a method for detecting very early atherosclerotic changes that generally occur decades before atherosclerosis becomes clinically evident. Hence, molecular imaging will provide a method for early detection and treatment of patients who are likely to have aggressive lesion growth.

Most forms of diagnostic medical imaging are based on the detection of pathologic changes in tissue morphology or function that occur late in the disease process. More recently, methods for detecting the underlying pathophysiologic cellular or molecular processes have been explored. The most common strategy has been to create novel targeted contrast agents that bind to disease-related antigens. Targeted molecular and cellular imaging may potentially improve patient care by detecting diseases at an early stage, guiding treatment strategy according to phenotype, and rapidly evaluating response to therapy.

For cardiovascular disease, molecular imaging could have a major clinical impact by detecting thrombus formation or early vascular pathophysiologic changes that contribute to the initiation of atherosclerotic disease and plaque instability. The ability to non-invasively assess the expression of adhesion molecules that participate in the recruitment of platelets, such as von Willebrand factor (vWF), or proteases that regulate the coagulation cascade, such as thrombin, could be used to gain a clearer understanding of kinetics of pathological thrombus development, to develop methods for identifying patients who are likely to have aggressive or unstable clot formation, and to test novel treatments aimed at modulating thrombosis.

Herein, novel contrast-enhanced ultrasound (CEU) molecular imaging methods for detecting vascular thrombi and atherosclerotic lesions that are thrombogenic and high risk for complications in clinically relevant models of disease are provided. Specifically, glycoprotein Ib (GPIb)-surface conjugated microbubble ultrasound contrast agents will be used to target the adhesive protein vWF and the coagulation protein thrombin. CEU with GPIb-microbubbles can be used to detect the presence of thrombus formation in large vascular compartments or in the microcirculation. Additionally, CEU with GPIb-microbubbles can be used to detect a prothrombotic endothelial phenotype in an animal model of severe atherosclerotic disease.

The interaction between the vulnerable atherosclerotic plaque and thrombus formation forms the basis of acute coronary syndromes, which represent a spectrum of ischemic myocardial events that share a similar pathophysiology. They include unstable angina, myocardial infarction, and sudden death. Normal endothelium plays a pivotal role in vascular homeostasis and limits the development of atherosclerosis. However, dysfunctional endothelial cells can change their activity substantially from their normal physiological state. For example, instead of forming a remarkably antithrombotic surface, dysfunctional endothelial cells develop prothrombotic activities with increased adhesiveness for platelets and leukocytes and secretion of procoagulant compounds leading to thrombin generation (Forgione et al. (2000) Curr. Opin. Cardiol., 15:409-415; Gimbrone et al. (1999) Am. J. Pathol., 155:1-5; Traub et al. (1998) Arterioscler. Thromb. Vasc. Biol., 18:677-685). There is also evidence that platelet interactions with endothelial cells, even brief interactions, serve as a source for deleterious pro-inflammatory cytokines, growth factors and vasoactive compounds (Huo et al. (2004) Trends Cardiovasc. Med., 14:18-22). The mechanism by which dysfunctional endothelial cells promotes platelet thrombosis involves two steps: 1) primary recruitment and adhesion of platelets; 2) secondary aggregation of platelets. Endothelial cells accumulate vWF within their Weibel-Palade bodies, which are secreted upon injury (Andre et al. (2000) Blood 96:3322-3328; Andrews et al. (2004) Thr. Res., 114:447-453; Ruggeri et al. (2002) Nat. Med., 8:1227-1234). vWF released onto the surface of dysfunctional endothelial cells represents a unique anchor for circulating platelets through the GPIb receptor. While the primary role of platelets is to trigger hemostasis in order to maintain vascular integrity, platelets are unable to differentiate between a disrupted vessel wall within, for example, a small digital vein and the atherosclerotic disruption of a coronary artery. As a consequence, the function of normal platelets is usually too efficient for the safety of patients with coronary artery disease, and potent antiplatelet drugs have been designed to reduce platelet function. However, early diagnosis and treatment is dependent upon robust techniques to detect dysfunctional endothelial cells and platelet deposition in patients prior to plaque rupture.

A more specific and sensitive method for the early detection of athero-prone regions and vascular clots in the vasculature is needed. An ideal approach would be to assess platelet accumulation or the adhesion molecules responsible for their recruitment. Thrombus formation at the moderate-to-high shear rates found within arterioles and diseased vascular beds requires an orchestrated series of receptor-mediated events facilitating platelet adhesion, rapid cellular activation, and the subsequent accumulation of fibrin and additional platelets into a growing hemostatic plug. Initial platelet deposition is triggered exposure of ECM proteins such as vWF. ECM-bound vWF plays a critical role in the tethering of platelets at high shear levels due to the rapid on-rate of binding between vWF and the platelet receptor GPIb (Andrews et al. (2004) Thr. Res., 114:447-453). The rapid off-rate of GPIb-vWF interactions results in platelet translocation at the site of injury (McCarty et al. (2006) J. Thromb. Haemost., 4:1367-1378), allowing adhesive interactions with slower binding kinetics (i.e. platelet receptors GPVI and/or α_{IIb}β₃ integrins) to mediate platelet adhesion following activation (Watson et al. (2005) J. Thromb. Haemost., 3:1752-1762). Subsequent platelet-platelet adhesion (aggregation) is predominately mediated by two receptors, GPIb and α_{IIb}β₃, with the contribution of GPIb becoming progressively more important with increasing blood flow. Under high shear, platelet-bound vWF is the major ligand promoting the tethering of platelets, while fibrinogen and thrombin play critical roles in maintaining clot stability. Importantly, it has recently shown that GPIb signaling following vWF binding is sufficient to mediate platelet activation and cytoskeletal reorganization (McCarty et al. (2006) J. Thromb. Haemost., 4:1367-1378). This has considerable implications seeing that platelet activation plays a crucial role in the process of hemostasis. However, in diseased vessels, platelet activation can result in vessel occlusion, leading to heart attack and stroke. As a result, endothelial vWF expression has attracted considerable interest as a predictor of cardiovascular disease (CVD). Given the key role of vWF in arterial thrombus formation, increased vWF expression levels contribute to a prothrombotic state and can be used as a predictor of adverse cardiovascular events.

Following vascular injury or plaque rupture, concomitant with platelet recruitment and activation are the first steps of blood coagulation, which are the exposure and activation of tissue factor and factor XII (Renne et al. (2006) Blood Cells Mol. Dis., 36:148-151; Renne et al. (2005) J. Exp. Med., 202:271-281; Steffel et al. (2006) Circulation 113:722-731). These two steps lead to the sequential activation of other coagulation factors into their corresponding active forms as serine proteases. Protease activation culminates with the generation of thrombin (Coughlin, S. R. (2005) J. Thromb. Haemost., 3:1800-1814; Mangin et al. (2006) Blood 107:4346-4353; Sambrano et al. (2001) Nature 413:74-78). Thrombin not only attracts and activates platelets and cleaves fibrinogen, which leads to fibrin production and clot formation, but also mediates the feedback activation of coagulation cofactors. This feedback mechanism leads to an autocatalytic cascade, resulting in rampant clot formation. During clot formation, thrombin is immobilized on the surface of the fibrin-rich clot (Becker et al. (1999) J. Biol. Chem., 274:6226-6233), thereby localizing thrombin to the site of vascular injury. Importantly, it has recently been shown that surface-immobilized thrombin is able to directly capture and activate platelets under shear flow conditions, and that this recruitment is critically dependent upon thrombin binding to platelet GPIb (Gruber et al. (2007) Blood; Thornber et al. (2006) FEBS J., 273:5032-5043). While thrombin generation plays a critical role in hemostasis at sites of injury, the rupture of an atherosclerotic plaque in a diseased vessel triggers thrombin generation and activation of the coagulation cascade, resulting in occlusive clots (Corti et al. (2002) Am. J. Med., 113:668-680). Moreover, one third of acute coronary syndromes, particularly sudden death, occur without full plaque rupture but rather superficial erosion of markedly stenotic and fibrotic plaque resulting in acute thrombin generation and localization. Therefore, surface-bound thrombin can be used as an early indicator of unstable and athero-prone plaque formation.

Since microbubble ultrasound agents are pure intravascular tracers, strategies to image vascular clots must rely on targeting disease-related markers within the vascular space. Potential targets include platelet surface markers that are only expressed upon platelet activation, and therefore include ligands for the unique platelet receptors GPIb and α_{IIb}β₃. Microbubbles have been successfully targeted to α_{IIb}β₃ in in vitro models under static conditions (Schumann et al. (2002) Invest. Radiol., 37:587-593), however in vivo targeting has been limited by the relatively low-affinity and low-specificity of the small peptide ligands. However, GPIb is the high affinity receptor for both vWF and thrombin. Accordingly, GPIb and fragments, derivatives, mutants, and variants thereof which retain GPIb binding activity, are more appropriate as a targeting moieties. In a particular embodiment, the mutant/variant/derivative/fragment of GPIb possesses increased VWF binding. For example, GPIb (His86Ala) (Peng et al. (Blood (2005) 106:1982-1987) has increased VWF binding affinity and can increase the residence time and strength of the GPIb coupled microbubble to VWF under flow. Additionally, the soluble form of GPIb (glycocalicin; see, e.g., Baglia et al. (J. Biol. Chem. (2004) 279:45470-45476) and Baglia et al. (J. Biol. Chem. (2004) 279:49323-49329)) or recombinant GPIb (see, e.g., Li et al. (Protein Expr. Purif. (2001) 22:200-210) may be conjugated to the microbubbles. In a particular embodiment, the targeting moieties of the instant invention may be linked to the microbubbles via specific binding pairs, such as an antigen-antibody. For example, anti-calmodium (CaM) mAb may be biotinylated and conjugated to the microbubbles via a streptavidin linker followed by incubation with recombinant GPIb-CaM, a chimeric protein (see, e.g., Li et al. (Protein Expr. Purif. (2001) 22:200-210), to link GPIb to the microbubbles.

Physiologically, GPIb mediates selective platelet recruitment to sites of vascular injury and atherosclerotic plaques under shear flow conditions. Importantly, GPIb-mediated platelet recruitment is one of the initial steps in the development of vascular clots, even prior to formation of occlusive clots or plaque rupture (Croce et al. (2007) Curr. Opin. Hematol., 14:55-61). Therefore, spatial localization of GPIb-microbubbles represents a potentially useful diagnostic tool to detect both acute and chronic thrombus development.

Contrast-enhanced ultrasound has been shown to be well-suited for the application of molecular and cellular imaging (see hereinabove and Christiansen et al. (2002) Circulation 105:1764-1767; Ellegala et al. (2003) Circulation 108:336-341; Leong-Poi et al. (2005) Circulation 111:3248-3254; Leong-Poi et al. (2003) Circulation 107:455-460; Lindner et al. (2000) Circulation 101:668-675; Lindner et al. (2000) Circulation 102:531-538; Lindner et al. (2000) Circulation 102:2745-2750). This methodology allows the conjugation via a long molecular polyethyleneglycol tether per of several thousand targeting ligands per square micron surface of each microbubble. Compared to most other imaging methods, CEU is well balanced in terms of sensitivity and spatial resolution, and is able to detect signals from a single microbubble (Klibanov et al. (2002) Acad. Radiol., 9:S279-281). At the same time, CEU has a resolution of under 1 mm. Spatial localization of signal enhancement can be further enhanced by fusion display in which contrast signal obtained at low to medium frequencies is superimposed on high-frequency, high frequency images (Kaufmann et al. (2007) J. Am. Soc. Echocardiogr., 20:136-143). The relative limitation of high background signal from tissue has been overcome with multi-pulse imaging techniques (pulse inversion, amplitude modulation, and power-Doppler imaging) that null the background tissue in combination with off-line background subtraction (Behm et al. (2006) Ultrasound Q., 22:67-72). The best-recognized advantages of CEU, however, are the widespread availability of ultrasound systems, the convenience and portability of ultrasound imaging equipment, and the ability to perform targeted imaging protocols in less than 15 minutes (Lindner, J. R. (2004) Nat. Rev. Drug Discov., 3:527-532). All of these characteristics make CEU attractive for clinical use, and its application in the research setting for high-throughput evaluation of new technologies.

### IV. IMAGING

Microbubbles are effective ultrasound agents due to an acoustic impedance mismatch between the microbubbles' encapsulated gas and the surrounding blood. Any means which can be used to detect this acoustic impedance mismatch is contemplated with the instant invention. Techniques for the detection of the microbubbles include, without limitation, magnetic resonance imaging (MRI; with or without conjugation of paramagnetic agents), optical imaging (e.g., optical coherence, near-infrared (NIR) conjugates), and photoacoustics (light stimulation and acoustic detection). In a particular embodiment, ultrasound techniques, such as contrast-enhanced ultrasound, are used to detect the microbubbles of the instant invention.

The following examples provide illustrative methods of practicing the instant invention, and are not intended to limit the scope of the invention in any way.

### Example 1

### Microbubbles Comprising rPGSL-Ig

The targeted microbubble contrast agent was prepared as follows. Biotinylated microbubbles were prepared by high-power sonication of a decafluorobutane bas-saturated aqueous suspension of distearoylphosphatidylcholine, polyoxyethylene-40-stearate, and distearoyl-phosphatidylethanolamine-PEG(2000)biotin. Microbubbles were washed by flotation centrifugation, exposed to streptavidin (30 μg per 10⁸ microbubbles), and washed. A recombinant P-selectin ligand composed of the amino terminal region of PSGL-1 in a selectin-binding glycoform fused to the Fc portion of human IgG1 (rPSGL-Ig) was conjugated to the microbubble (Y's Therapeutics, Burlingame Calif.). For this process, the Ig portion of the ligand was biotinylated. Microbubbles were then exposed to the biotinylated rPSGL-Ig (50 μg per 10⁸ microbubbles), then washed. Microbubble size and concentration were measured by electrozone sensing (Multisizer III, Beckman-Coulter, Fullerton, Calif.). Selective attachment of these microbubbles to P-selectin in variable shear conditions has been tested in flow chamber studies. They appear to have equivalent binding capabilities as monoclonal antibody-based targeting. Intravital microscopy studies of surgical trauma-induced P-selectin expression also demonstrated equivalent binding for the two preparations in a murine model. Targeted contrast enhanced ultrasound imaging has demonstrated selective attachment of rPSGL-Ig-bearing microbubbles in muscle tissue exposed to either TNF-alpha or ischemia-reperfusion injury. It has been demonstrated that microbubbles targeted via surface conjugation of rat mAb for mouse P-selectin can detect recent myocardial ischemia in mice. The use of rPSGL as a targeting moiety will provide a method to target P-selectin in any species including humans.

The microvascular behavior of microbubbles in postischemic muscle was assessed by intravital microscopy. The cremaster muscle of anesthetized mice was exteriorized, placed on a custom-made stage and observed with microscopy during isothermic superfusion. 5 mice were subjected to 20 minutes of cremasteric ischemia achieved by compression of the muscle's vascular pedicle, followed by 45 minutes of reperfusion. P-Selectin targeted and control microbubbles were then injected simultaneously. After allowing 10 minutes for circulation, microbubble attachment was quantified with dual filter fluorescent microscopy. The same experiment was performed in 4 mice not subjected to ischemia-reperfusion at an identical timepoint after surgical preparation.

For molecular imaging after acute myocardial ischemia reperfusion, mice were anesthetized and ventilated. In 11 mice, the LAD was exposed with a thoracotomy and occluded for 10 minutes with a suture. In 4 animals, a sham operation was performed. Myocardial perfusion and wall motion were assessed during ischemia. After 45 minutes of reperfusion, targeted myocardial contrast echocardiography was performed and myocardial perfusion and wall motion were reassessed. In 3 mice, targeted myocardial contrast echocardiography was performed without a thoracotomy.

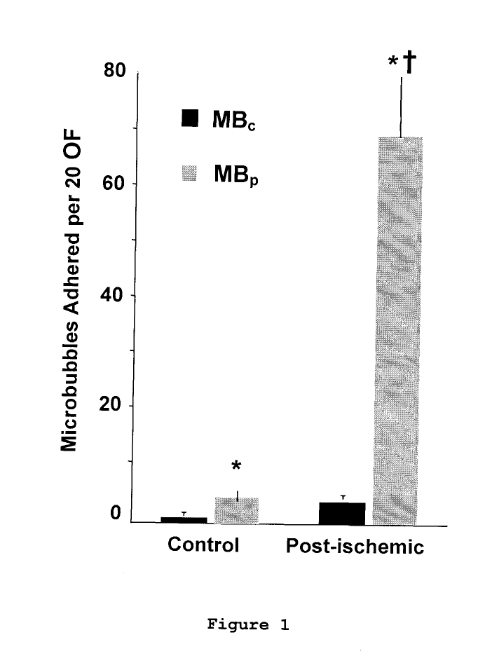

As seen in FIG. 1, there is a marked increase in retention of P-selectin targeted microbubbles in mice undergoing ischemia reperfusion.

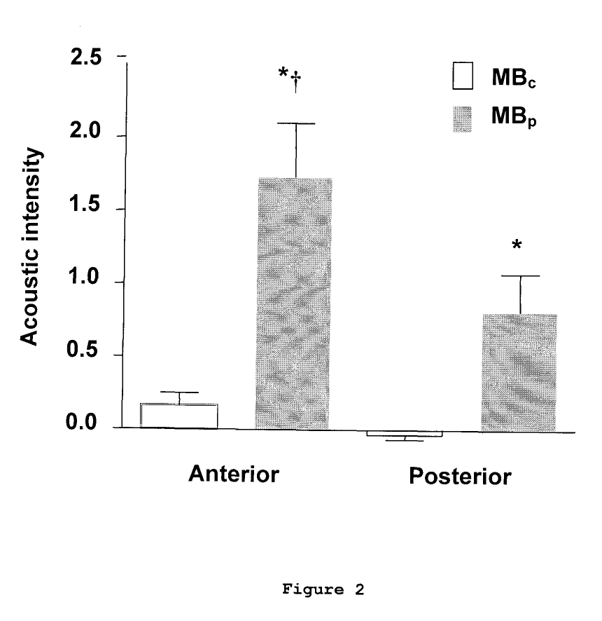

In a standard preparation of microbubbles, 3-6% of the microbubbles are greater than 5 μm in diameter. With size segregation, the microbubble preparation may consist of less than 0.1% microbubbles with a diameter greater than 5 μm. To avoid potential size dependent microbubble lodging, size segregated microbubbles were used in myocardial ischemia-reperfusion experiments with an additional 6 mice. With these preparations, the signal from control microbubbles (MB_{c}) was virtually eliminated. Again, the anterior and the posterior myocardium showed a significantly larger signal from P-selectin microbubbles (MBₚ) (FIG. 2).

Accordingly, P-Selectin expression in post-ischemic myocardium can be imaged with targeted myocardial contrast echocardiography at a time when myocardial perfusion and wall motion have returned to normal. Thus, molecular imaging of P-selectin expression may be effective in risk stratifying patients with chest pain.

### Example 2

### Comparative Studies

### Materials and Methods

### Preparation of Microbubbles

Microbubbles with monoclonal antibodies against P-selectin (MB_{AB}); isotype control antibodies (MB_{c}); or rPSGL-Ig (Y's Therapeutics; Tokyo, Japan) (MB_{PGSL}) conjugated to their surfaces were created. Biotinylated microbubbles containing decafluorobutane gas were prepared as previously described (Klibanov et al. (1999) Proc. 26th Intl. Symp. Controlled Rel. Bioact. Mat. 124-125). Approximately 3×10⁸ biotinylated microbubbles were incubated for 30 minutes with 90 μg streptavidin (Sigma) and washed. Aliquots of the suspension (1×10⁸ microbubbles) were incubated for 30 minutes with 75 μg of biotinylated (EZ-Link, Pierce, Rockford, Ill.) rat anti-mouse monoclonal IgG1 against P-selectin (RB40.34) or isotype control antibody (R3-34, Pharmingen Inc., San Diego, Calif.). The antibody concentration used was determined by flow cytometry experiments.

### Flow Chamber Studies

The in vitro binding capability of MB_{PSGL} and MB_{Ab} was tested with a parallel plate flow chamber with a P-selectin density of 100 molecules/mm² at various shear stresses. More specifically, attachment was assessed at shear stresses of 1, 2 and 8 dynes/cm² (n=2 plates per shear stress level). The flow chamber was continuously perfused at appropriate flow rates for each wall shear stress level with isotonic phosphate buffered saline containing 3% BSA to which a mixture of MB_{PSGL} and MB_{Ab} each at a concentration of 3×10⁶/ml was added. After allowing 5 minutes of perfusion for microbubble adherence, the number of MB_{PSGL} and MB_{Ab} adhered to the flow chamber per optical field were counted and expressed as a retention fraction.

### Animal Preparation

The study protocol was approved by the Animal Research Committee at the University of Virginia. Mice were anesthetized with an injection (12.5 μL/g IP) of a solution containing ketamine hydrochloride (10 mg/mL), xylazine (1 mg/mL), and atropine (0.02 mg/mL). Body temperature was maintained at 37° C. with a heating pad. Both jugular veins were cannulated for administration of microbubbles and drugs.

### Intravital Microscopy

For direct in vivo observation of microbubble attachment to inflamed endothelium, intravital microscopy of mouse cremasteric muscle was performed in 3 mice. Inflammation of the cremaster muscle may be produced by intrascrotal injections of 0.5 μg murine tumor necrosis factor (TNF)-α (Sigma, St. Louis, Mo.) 2 hours. P-selectin expression was induced by surgical exposure of the cremaster muscle which was confirmed by leukocyte rolling in all observed venules. DiI-labeled MB_{PSGL} and DiO-labeled MB_{Ab} (5×10⁶ for each) (for labeling, see, e.g., Lindner et al. (2000) Circulation 102:2745-2750) were simultaneously injected via a jugular catheter. Microscopy was performed with combined fluorescent epi-illumination (460- to 500-nm excitation filter) and low-intensity transillumination. The number of microbubbles adherent in venules was determined in non-overlapping optical fields 10 minutes after injection using excitation filters for DiI and DiO (530 and 490 nm, respectively).

### Targeted Imaging of Inflammation

Targeted signal from MB_{PSGL}, MB_{Ab}, and MB_{c} microbubbles was assessed by contrast-enhanced ultrasound (CEU) of proximal hindlimb adductor muscle undergoing ischemic injury. Imaging was performed in either: a) wild type mice undergoing ischemic injury (n=6); b) genetically modified P-selectin-deficient (P^{-/-}; see, e.g., Bullard et al. (1995) J. Clin. Invest. 95:1782-1788) mice undergoing ischemic injury (n=6); and c) non-ischemic wild-type control mice (n=4). Proximal hindlimb ischemia was produced by 8 minute external band occlusion of the limb feeding arterial supply. Imaging was performed beginning 45 minutes after reperfusion. For each imaging study 3×10⁶ MB_{PSGL}, MB_{Ab}, or MB_{c} were injected intravenously in random order. As previously described (see, e.g., Lindner et al. Circulation (2001) 104:2107-2112), an image reflecting only retained microbubbles was derived by acquiring the initial frame at 8 minutes after microbubble injection and then digitally subtracting subsequent averaged frames at a long pulsing interval (10 seconds) that were obtained after several seconds of continuous high-power imaging.

### Results

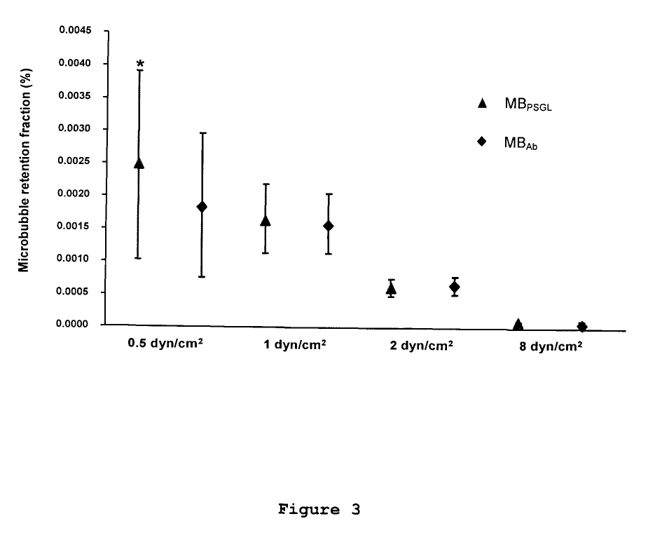

In flow chamber experiments, attachment to P-selectin for both MB_{PSGL} and MB_{Ab} decreased with increasing shear stress. Microbubble retention fraction was equivalent for MB_{PSGL} and MB_{Ab} at all except the lowest (0.5 dynes/cm²) wall shear stress, at which MB_{PSGL} showed a small but statistically significant (p=<0.05) increase in adherence (FIG. 3).

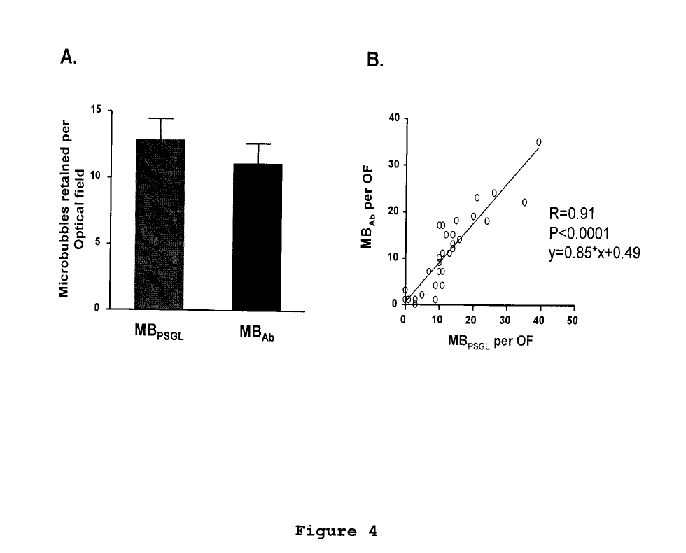

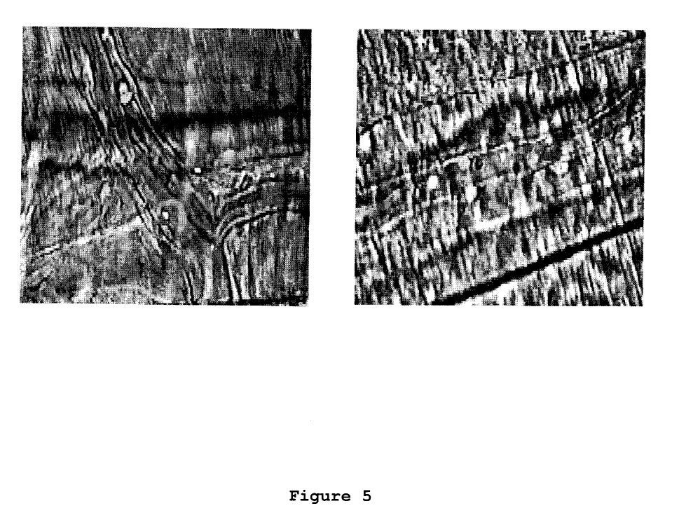

On intravital microscopy, P-selectin expression from surgical preparation resulted in leukocyte rolling in all venules observed. Venular endothelial attachment was similar for MB_{PSGL} and MB_{Ab} (FIG. 4A). Despite a wide range of microbubbles adhesion between optical fields (retention heterogeneity), there was a good correlation between the number of MB_{PSGL} and MB_{Ab} which adhered for a given optical field (FIG. 4B). Pseudocolorized images from intravital microscopy illustrating microbubble adherence to small venules are shown in FIG. 5. Images were generated by superimposition of individual images with separate fluorescent filters for DiI-labeled MB_{Ab} (red) and DiO-labeled MB_{PSGL} (green).

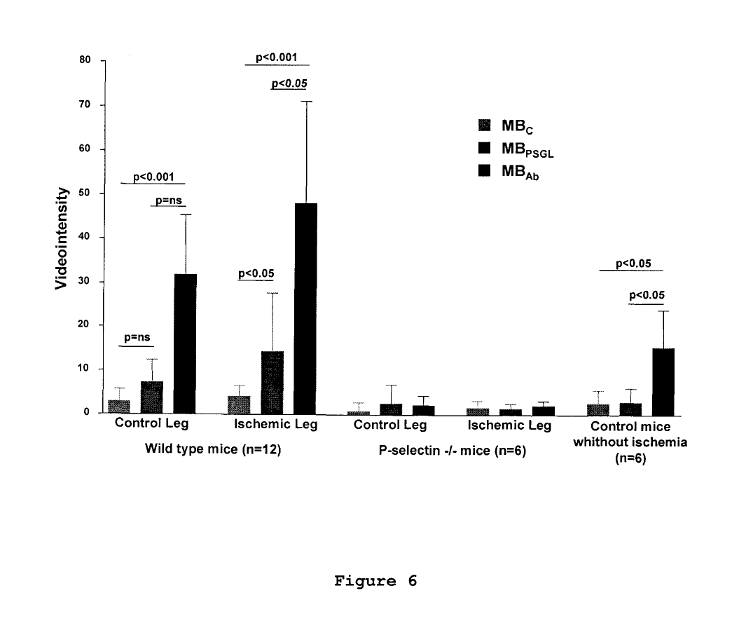

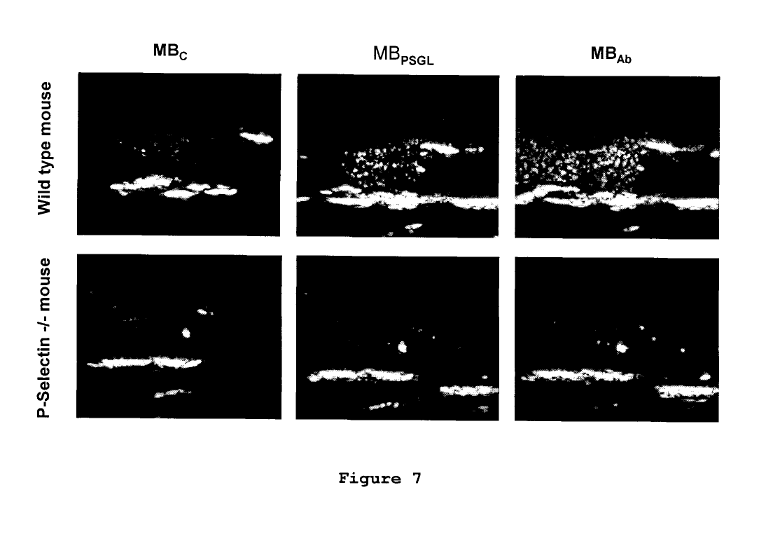

In wild type animals undergoing ischemia reperfusion injury, mean (+SD) signal intensity in the post-ischemic hindlimb incrementally increased for MB_{c}, MB_{PSGL}, and MB_{Ab} (FIG. 6). Significant signal enhancement was also seen in the contralateral control leg. In P^{-/-}mice undergoing ischemia-reperfusion injury, signal enhancement was similarly low for all microbubbles in both limbs. In control non-ischemic wild-type animals, signal from MB_{Ab} was significantly and undesirably elevated compared to that from MB_{C} and MB_{PSGL}. Accordingly, the degree of signal enhancement due to ischemia in wild type mice (ratio of signal from the post-ischemic limb to that in non-ischemic controls) was substantially greater for MB_{PSGL} than for MB_{Ab} (4.9- vs 3.1-fold). Illustrative images from targeted contrast-enhanced ultrasound are shown in FIG. 7.

In view of the above, a bioengineered form of the natural P-selectin ligand PSGL-1 can be used for contrast-enhanced ultrasound molecular imaging of inflammation. This strategy provides comparable levels total enhancement compared to antibody targeting and significantly greater specificity due to very low specific attachment in normal tissue. Clearly, microbubbles bearing a PSGL-1 analog are an effective and safe means for diagnostic molecular imaging in animals, including humans.

### Example 3

### Microbubbles with VCAM-1

Atherosclerosis is a chronic inflammatory disorder that often progresses silently for decades before becoming clinically evident (Ross R.(1999) N Engl J Med 340:115-26). In current clinical practice, C-reactive peptide is the only inflammatory marker routinely used for risk assessment in patients. Non-invasive imaging of vascular changes such as coronary calcification, carotid intimal-medial thickening and plaque morphology have recently been used to assess patient risk (Arad et al. (2000) J. Am. Coll. Cardiol., 36:1253-60; Greenland et al. (2004) JAMA 291:210-5; Chambless et al. (2000) Am. J. Epidemiol., 151:478-87; O'Leary et al. (1999) N. Engl. J. Med., 340:14-22; Leber et al. (2006) J. Am. Coll. Cardiol., 47:672-7). However, these methods detect changes that occur relatively late in the disease process and do not directly assess inflammatory status. Since inflammation participates in plaque initiation and progression, a method capable of imaging the extent of vascular inflammation could potentially provide powerful predictive information on both early disease presence and future risk for disease progression. At latter stages of disease, it could also provide information on plaque vulnerability to erosion and rupture (Virmani et al. (2006) J. Am. Coll. Cardiol., 47:C13-C18). It is also important to recognize that new therapies aimed at inhibiting vascular inflammatory responses are being developed and will likely be most effective when used in conjunction with quantitative methods that can detect early inflammatory changes.

Vascular cell adhesion molecule-1 (VCAM-1) is expressed by activated endothelial cells and participates in leukocyte rolling and adhesion primarily by interacting with its counterligand VLA-4 (α₄β₁) on monocytes and lymphocytes (Carlos et al. (1991) Blood 77:2266-71; Huo et al. (2000) Circ. Res., 87:153-9). VCAM-1 expression on the vessel endothelial surface or the underlying vasa vasorum plays an important role in atherosclerotic plaque development by monocyte and T-lymphocyte recruitment (O'Brien et al. (1996) Circulation 93:672-82). It is an ideal target for molecular imaging because there is little constitutive expression and its upregulation occurs at the very earliest stages of atherogenesis (Nakashima et al. (1998) Arterioscler. Thromb. Vasc. Biol., 18:842-51; Iiyama et al. (1999) Circ. Res., 85:199-207). Molecular imaging of VCAM-1 with targeted contrast-enhanced ultrasound (CEU) could be used to evaluate the degree of vascular inflammation in atherosclerosis. CEU is well-suited for such screening purposes due to practical considerations such as cost, short duration of imaging protocols (10 minutes), and balance between spatial resolution and sensitivity for targeted contrast agent detection. To test the above hypothesis, attachment of VCAM-1-targeted microbubbles to endothelial cells was evaluated under variable shear conditions. Microbubble attachment in vivo and signal enhancement of the aorta was assessed in animal models of varying degrees of atherosclerosis produced by dietary intervention in wild-type and Apolipoprotein-E-deficient (ApoE^{-/-}) mice.

### Methods

### Microbubble Preparation

Biotinylated, lipid-shelled decafluorobutane microbubbles were prepared by sonication of a gas-saturated aqueous suspension of distearoylphosphatidylcholine, polyoxyethylene-40-stearate and distearoylphosphatidylethanolamine-PEG(2000)biotin. Rat anti-mouse monoclonal IgGl against VCAM-1 (MK 2.7) or isotype control antibody (R3-34, Pharmingen Inc.; SAn Diego, Calif.) were conjugated to the surface of microbubbles as previously described to produce VCAM-1-targeted (MB_{V}) or control (MB_{c}) microbubbles (Lindner et al. (2001) Circulation 104:2107-12). For flow-chamber and in vivo attachment studies, microbubbles were fluorescently labeled by the addition of either dioctadecyltetramethylindocarbocyanine (DiI) or dioctadecyloxacarbocyanine (DiO) perchlorate (Molecular Probes Inc.; Eugene, Oreg.) to the aqueous suspension. Microbubble concentrations were measured by electrozone sensing (Multisizer III, Beckman-Coulter; Fullerton, Calif.).

### Flow-Chamber Adhesion Studies

Murine endothelial cells (SVEC4-10, ATCC) that express VCAM-1 were grown to confluence in DMEM supplemented with 10% fetal bovine serum on fibronectin-coated culture dishes (Sasaki et al. (2003) Am. J. Physiol. Cell Physiol., 284:C422-C428). For activation, cells were pre-treated with TNF-α (20 ng/mL) for 4 hours. Culture dishes were mounted on a parallel plate flow chamber (Glycotech; Gaithersburg, Md.) with controlled gasket thickness and a channel width of 2.5 mm. The flow chamber was placed in an inverted position on a microscope (Axioskop2-FS, Carl Zeiss Inc.; Thornwood, N.Y.) with a ×40 objective and high-resolution CCD camera (C2400, Hamamatsu Photonics; Bridgewater, N.J.) for video recording. A suspension of control or VCAM-1-targeted microbubbles (3×10⁶ ml⁻¹) in cell culture medium was drawn through the flow chamber with an adjustable withdrawal pump. The number of microbubbles attached to cells was determined for 20 optical fields (total area 0.5 mm²) after 5 minutes of continuous flow at rates to generate shear rates of 0.5 to 12.0 dyne/cm². Experiments were performed in triplicate as a minimum. Since aortic flow is pulsatile, adhesion at the highest shear rates (8 and 12 dyne/cm², n=6 for each) was also assessed after transient (5 seconds) reductions of shear to <0.5 dyne/cm². This duration was the minimum required for significant flow reduction due to the capacitance of the flow chamber system. Three sequential flow reductions were performed after 5 minutes of continuous flow and microbubble attachment after each was determined once shear had returned to pre-pause levels.

### Animal Models and Preparation

The study protocol was approved by the institutional Animal Research Committee. 26 male wild-type C57B1/6 and 23 ApoE^{-/-}mice (Jackson Laboratory; Bar Harbor, Me.) were studied at 22-24 weeks of age. Mice were fed either chow diet or, from 14 weeks of age onwards, a hypercholesterolemic diet (HCD) containing 21% fat by weight, 0.15% cholesterol, and 19.5% casein without sodium cholate. Anesthesia was induced with an intraperitoneal injection (12.5 μL·g⁻¹) of a solution containing ketamine hydrochloride (10 mg·mL⁻¹), xylazine (1 mg·mL⁻¹) and atropine (0.02 mg·mL⁻¹). A jugular vein was cannulated for administration of microbubbles.

### Assessment of Microbubble Attachment to the Aorta

In anesthetized mice, VCAM-1-targeted and control microbubbles (1×10⁶ for each) labeled with DiI and DiO, respectively, were injected simultaneously by intravenous route. After 10 minutes, a right atriotomy incision was made through an anterior thoracotomy. The blood volume was removed with 10 mL of 5% bovine serum albumin containing heparin at 35-37° C. infused via a left ventricular puncture at an infusion pressure ≦100 mm Hg. The aorta was removed, a longitudinal incision was made, and the aorta was pinned flat on a microscopy platform. En face microscopy observations of the ascending, arch and descending portions of the thoracic aorta were made with a ×20 objective. A minimum of 10 optical fields were observed under fluorescent epi-illumination at excitation wavelengths of both 490 and 530 nm.

### Contrast Enhanced Ultrasound Imaging

Ultrasound imaging (Sequoia, Siemens Medical Systems) was performed with a high-frequency linear-array probe held in place by a railed gantry system. The aortic arch and proximal descending aorta arch was imaged from a left parasternal window using fundamental imaging at 14 MHz to optimize the imaging plane in the longitudinal axis. CEU was performed with Contrast Pulse Sequencing™, which detects the non-linear fundamental signal component for microbubbles. Imaging was performed at a centerline frequency of 7 MHz and a mechanical index of 0.2. The gain was set just below visible speckle at baseline and held constant. Real-time imaging was performed 10 minutes after intravenous injection of 1×10⁶ MB_{C} or MB_{V}, performed in random order. After several seconds of continuous imaging at a mechanical index of 0.2, microbubbles in the sector were destroyed by increasing the mechanical index to 1.0 for 1 second. Subsequent post-destruction images were acquired at a mechanical index of 0.2. To determine signal from retained microbubbles alone, several post-destruction contrast frames representing freely circulating microbubbles were averaged and digitally subtracted from several averaged pre-destruction frames (Lindner et al. (2001) Circulation 104:2107-12). Background-subtracted intensity was measured from a region-of-interest placed over the aorta using the 14 MHz image as a guide.

Since microbubble attachment is dependent upon contact with the aortic wall, the axial distribution of microbubbles immediately after injection was assessed in 3 wild-type mice. Imaging was performed with an ultra-high frequency (30 MHz) mechanical sector imaging system (Vevo 770, Visualsonics Inc.) during an intravenous injection of MB_{C} (1×10⁶). Ultrasound was transmitted with one-cycle pulses with an axial resolution of 55 μm. Images were aligned and displayed as a maximum-intensity projection for 3 seconds after microbubble appearance.

### Measurement of Ultrasound Pressure Profile

Acoustic pressures within the imaging sector were measured in a water bath with a needle hydrophone (PVDF-Z44, Specialty Engineering Associates) coupled with an oscilloscope (TDS-3012, Tektronix Inc.; Beaverton, Oreg.). Peak negative acoustic pressure measurements were made at the focal depth using the system settings for targeted imaging. A 2-dimensional pressure profile was obtained by making 0.5 mm adjustments in the in-plane lateral dimension (beam width) and elevational dimension (beam thickness).

### Echocardiography

The peak flow velocity at the mid-arch was measured by pulsed-wave Doppler with a gate size at the minimum setting. Left ventricular systolic function was assessed by imaging in the short-axis plane at the mid-papillary muscle level with fundamental imaging at 14 MHz. Fractional shortening in the anterior-posterior and septal-lateral dimensions were measured by video calipers and averaged.

### Immunohistology

Immunostaining for VCAM-1 was performed on paraffin-embedded sections of the proximal and distal aortic arch after microwave treatment with Antigen Unmasking Solution (Vector Laboratories; Burlingame, Calif.) for several animals in each group. Goat polyclonal antibody to human VCAM-1 with cross-reactivity for mouse VCAM-1 (sc1504, Santa Cruz Biotechnology Inc.; Santa Cruz, Calif.) was used as a primary antibody with a biotinylated secondary anti-goat antibody (Vector Laboratories). Staining was performed using a peroxidase kit (ABC Vectastain Elite, Vector Laboratories) and 3,3′-diaminobenzidine chromagen (DAKO). Slides were counterstained with hematoxylin.

### Statistical Methods

Unless otherwise specified, parametric data are expressed as mean (±1 SD). Comparisons between microbubble agents within groups were performed by paired Student's T-test. Comparisons between multiple groups were performed with ANOVA and a Tukey post hoc test or, when appropriate, with a Kruskal-Wallis test with Dunn's post-hoc test. Differences were considered significant at p<0.05 (2-sided).

### Results

### Microbubble Attachment to Endothelial Cells In Vitro

Both non-activated and activated cultured SVECs stained positive for VCAM-1 on immunohistochemistry.

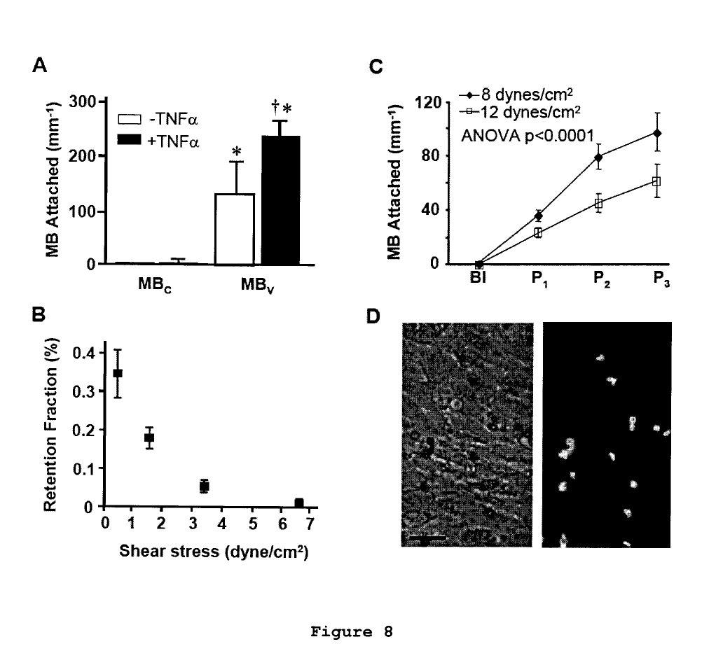

During flow-chamber studies at the lowest shear rate (0.5 dyne/cm²), there was minimal attachment of control microbubbles (MB_{C}) to SVECs irrespective of activation status (FIG. 8A). VCAM-1-targeted microbubbles (MB_{V}) attached to both non-activated and activated SVECs, with slightly more attachment to activated cells. Attachment of MBV to activated SVECs decreased with increasing shear rate (FIG. 8B). Little microbubble attachment occurred at continuous shear rates that exceeded 6 dyne/cm². However, sequential brief reductions in shear allowed VCAM-1-targeted microbubbles to permanently bind at the highest shear rates tested (8 and 12 dyne/cm²) (FIG. 8C), indicating the ability of microbubbles to firmly attach in the face of high shear when flow occurs in pulsatile rather than continuous conditions.

### Attachment of Microbubbles to the Aorta

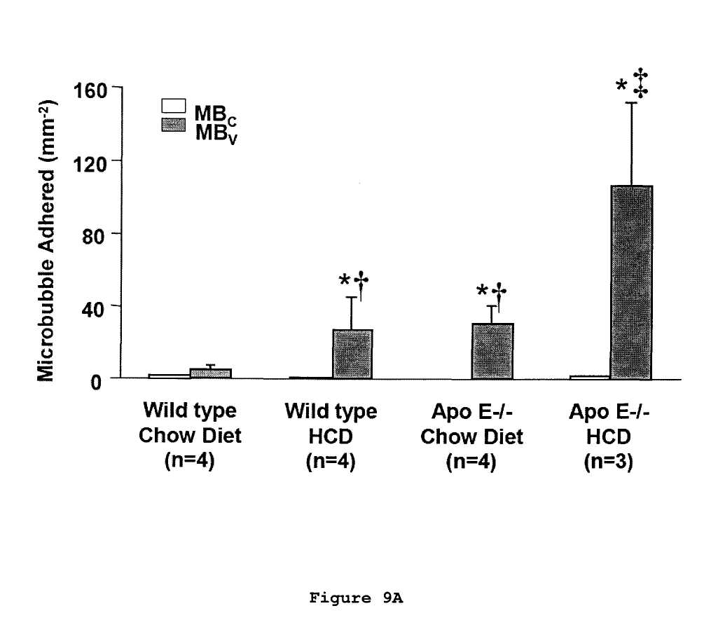

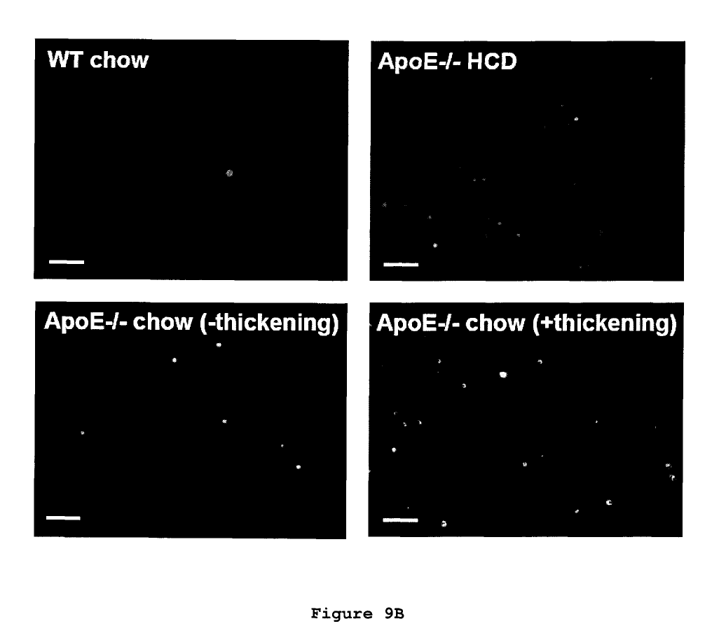

Ex vivo fluorescent microscopy of thoracic aortas removed 10 minutes after intravenous microbubble injection demonstrated little attachment for either control or VCAM-1-targeted microbubbles in wild-type mice on chow diet (FIG. 9). In the other groups (wild-type mice on HCD, and ApoE−/− mice on either chow or HCD), attachment of VCAM-1-targeted microbubbles to the aorta was greater than for control microbubbles. Attachment of VCAM-1-targeted microbubbles was significantly greater in ApoE^{-/-}mice on HCD than in any other group and was distributed throughout the aorta. In contrast, in ApoE^{-/-}mice on chow diet VCAM-1-targeted microbubbles tended to attach preferentially to regions of the aorta where there was irregular thickening consistent with atherosclerotic lesion development.

### Targeted Imaging of VCAM-1 Expression

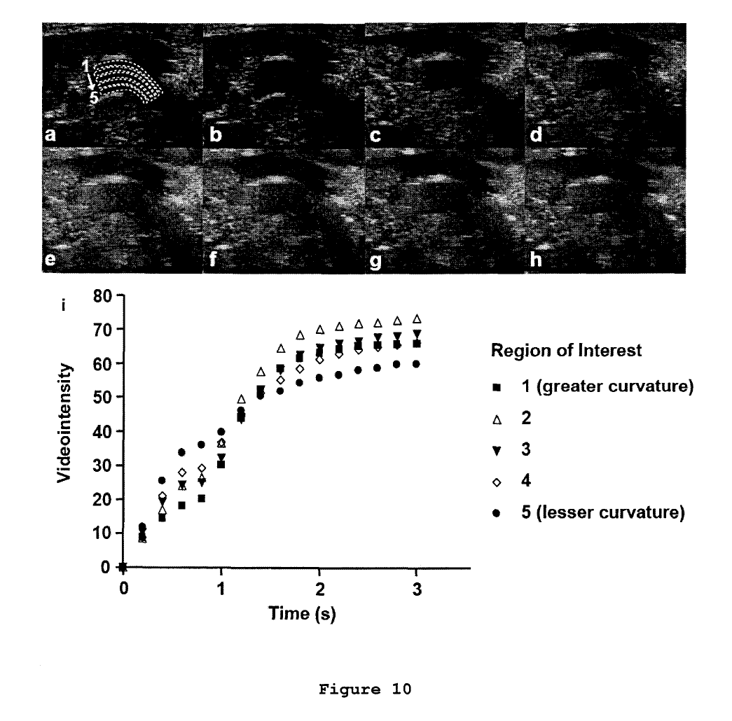

There were no significant differences between groups for left ventricular fractional shortening, peak systolic flow velocity in the aorta, or aortic diameter at the mid-arch (Table 1), indicating no systematic differences in hemodynamic conditions in the aortic arch. Flow velocities in the aortic arch reached near zero at end-diastole in most animals. CEU with ultra-high frequency (30 MHz) maximum-intensity projection demonstrated that the axial distribution of non-targeted microbubbles during their transit through the aorta extended to regions directly adjacent to the aortic wall in both the greater and lesser curvature of the arch (FIG. 10).

TABLE 1
Echocardiographic and Vascular Ultrasound Characteristics

| | Wild-type | Wild-type | ApoE−/− | ApoE−/− |
|---|---|---|---|---|
| | Chow diet | HCD | Chow diet | HCD |
| Aortic peak | 0.53 ± 0.10 | 0.52 ± 0.7 | 0.46 ± 0.13 | 0.50 ± 0.14 |
| velocity (m/s) | | | | |
| Fractional | 0.35 ± 0.04 | 0.35 ± 0.05 | 0.34 ± 0.05 | 0.38 ± 0.04 |
| shortening (%) | | | | |
| Aortic | 1.2 ± 0.2 | 1.4 ± 0.1 | 1.3 ± 0.2* | 1.4 ± 0.0.3 |
| diameter (mm) | | | | |

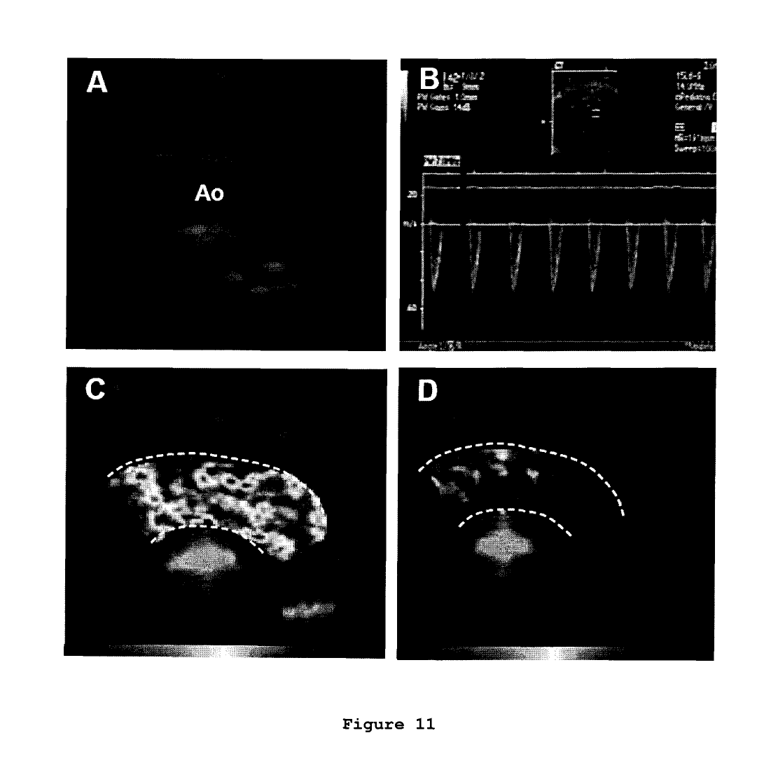

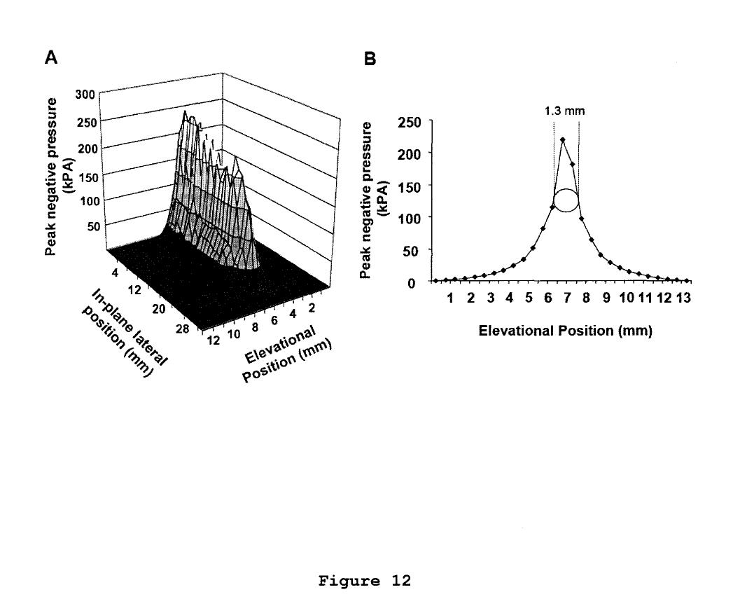

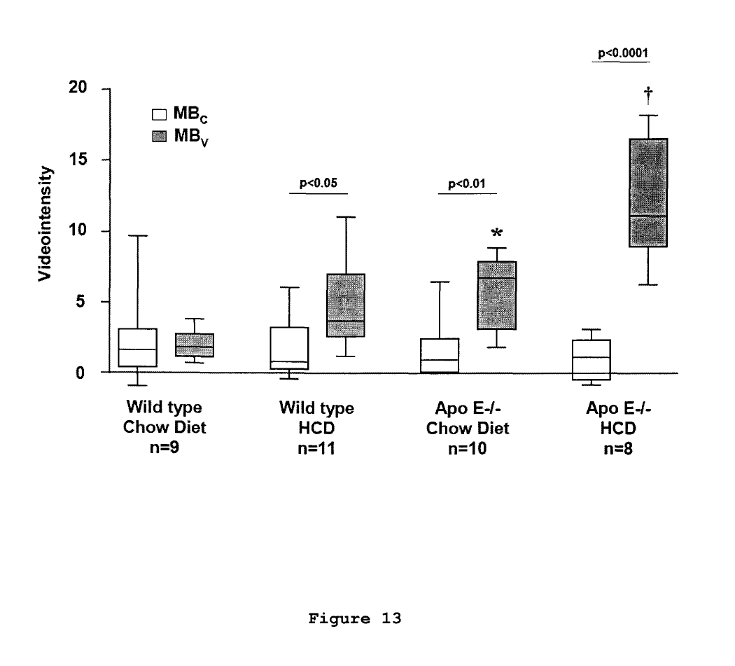

Illustrative B-mode, pulsed-wave Doppler, and background-subtracted color-coded CEU images from a single ApoE^{-/-} mouse on HCD are shown in FIG. 11. Strong signal enhancement was observed for VCAM-1-targeted but not control microbubbles. The profile of the peak negative acoustic pressures at the acoustic focus for the transducer and settings used for targeted CEU are illustrated in FIG. 12. According to the dimensions of the elevational plane, the entire volume of the aortic arch would be exposed to a peak negative acoustic pressure of ≧120 kPa before accounting for attenuation, and ≧96 kPa after correcting for attenuation assuming a coefficient of 1.1 dB/mm/MHz (FIG. 12) (Teotico et al. (2001) IEEE Trans. Ultrason. Ferroelectr. Freq. Control 48:593-601). These data indicate that the entire circumference of the aorta (circle on FIG. 12B) would fit in the effective detection profile of elevational plane. Hence, elevation plane averaging would permit detection of microbubbles attached to the front or back wall that were seemingly “out-of-plane” and explains the appearance of targeted stationary microbubble signal in the center of the apparent “lumen” in FIG. 11. FIG. 13 summarizes CEU data for all groups. Signal enhancement for control microbubbles was low and similar between groups. In wild-type mice on chow diet, signal for VCAM-1-targeted microbubbles was low and similar to that for control microbubbles. In contrast, in all other groups there was greater signal enhancement for VCAM-1-targeted compared to control microbubbles.

Signal enhancement for VCAM-1-targeted microbubbles incrementally increased from wild-type mice on HCD, to ApoE^{-/-} mice on chow, to ApoE^{-/-} on HCD.

### Immunohistochemistry

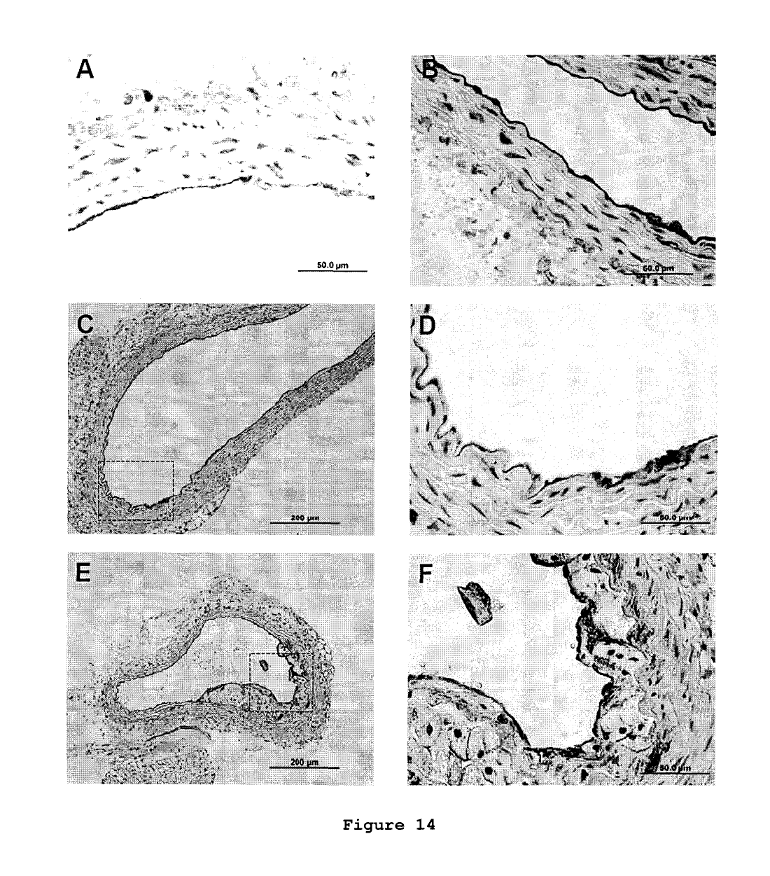

On histology there was no evidence for plaque development in wild-type mice irrespective of diet. On immunostaining, however, VCAM-1 expression was detected on the luminal endothelial surface of the aorta in wild-type mice on HCD (FIG. 14). In ApoE^{-/-} mice, there was intimal thickening and large atherosclerotic plaques protruding into the lumen, particularly in animals on HCD. Immunohistochemistry in ApoE^{-/-} mice demonstrated dense VCAM-1 expression on the endothelium, particularly overlying regions of plaque development. There was also VCAM-1 staining of neointimal monocytes, which are not accessible to microbubbles that are confined to the intravascular compartment. The degree of VCAM-1 staining on cells in the neointima qualitatively correlated with the degree of endothelial staining, and was more robust in ApoE^{-/-} mice when fed an HCD.

### Example 4

### Microbubbles Comprising GPIb

### Microbubble Preparation

Biotinylated, lipid-shelled decafluorobutane microbubbles will be prepared by sonication of a gas-saturated aqueous suspension of distearoylphosphatidylcholine, polyoxyethylene-40-stearate and distearoylphosphatidyl-ethanolamine-PEG(2000)biotin. The biotinylated soluble form of GPIb (glycocalicin), non-active mutant form of GPIb (deleted Cys209-Cys248 disulfide loop of GPIba), or recombinant GPIb, either whole or active-site fragments (e.g., fragments which retain similar binding properties of GPIb), will be conjugated to the surface of microbubbles using a streptavidin link (Lindner et al. (2000) Circulation 101:668-675; Lindner et al. (2000) Circulation 102:531-538; Lindner et al. (2000) Circulation 102:2745-2750). This conjugation will result in several thousand ligands per μm² shell surface area. GPIb density on the microbubble surface will be determined via flow cytometry with fluorescently-labeled anti-GPIb mAbs. For flow-chamber attachment studies, microbubbles will be fluorescently labeled by the addition of either DiI or DiO to the aqueous suspension. Microbubble concentration will be measured by electrozone sensing (Multisizer III, Beckman-Coulter. The average diameter for targeted microbubbles will be about 2-3 μm.

### Flow-Chamber Studies

A solution of vWF (10 μg/mL) or thrombin (1 U/ml) will be placed on culture dishes overnight at 4° C. then blocked with denatured BSA. Conformational activation of vWF will be performed by a 10 minute exposure to botrocetin (2 μg/mL). Dishes will be mounted on a parallel plate flow chamber (Glycotech) with controlled gasket thickness and a channel width of 2.5 mm. The flow chamber will be placed in an inverted position on a microscope (Axioskop2-FS, Carl Zeiss Inc.) with a high-resolution CCD camera (C2400, Hamamatsu Photonics) for video recording. A suspension of GPIb-labeled or control microbubbles (3×10⁶ ml⁻¹) will be drawn through the flow chamber with an adjustable withdrawal pump. The number of microbubbles attached to plates will be determined for 20 optical fields (0.5 mm²) after 5 minutes at flow rates to generate shear rates of 0.5 to 12.0 dyne/cm². The kinetics of GPIb-microbubble binding to vWF or thrombin will be calculated by recording the tethering rates and rolling velocities of GPIb-microbubbles at a range of shear rates.

### Intravital-Microscopy Studies

Attachment of targeted or control microbubbles in the microcirculation will be evaluated by intravital microscopy. The cremaster muscle of anesthetized mice will be exteriorized and secured to a custom microscopy pedestal during isothermic buffered superfusion. Intravital microscopy (Axioskop2-FS, Carl Zeiss Inc.) of the microcirculation will be performed. A 30-50 μm arteriole or venule will be punctured using a glass micropipette positioned with a stage micromanipulator (Narishige; East Meadow, N.Y.)(Christiansen et al. (2002) Circulation 105:1764-1767). One minute after thrombus formation, DiI-labeled GPIb- or DiO-labeled control microbubbles will be injected intravenously (5×10⁷ each). The number of microbubbles attached will be determined by dual-fluorescent epi-illumination. The flow and shear rates for the vascular segment will be determined from data on vessel diameter with calibrated videocalipers and centerline velocity made with a dual-slit photodiode. Up to 3 separate vascular punctures will be performed for each animal 20 minutes apart.

### Imaging of Vascular Thrombus

Poly-filament 5-0 silk suture will be soaked in human thrombin (5 μg/mL). In anesthetized rats, the thread will be percutaneously placed through the LV apex into the ventricular lumen through a 23 g needle guided by an ultrasound biomicroscopy/microinjection system (Vevo 770, VisualSonics, Inc). The external portion of the suture will be tied to secure in place and trimmed. Beginning 15 minutes after thread placement, targeted CEU imaging (7 MHz CPS non-imaging, Siemens Ultrasound) of the left ventricular (LV) cavity will be performed 10 minutes after intravenous injection of control or GPIb-microbubbles in random order. Imaging will be repeated 1 hour later. Immunohistochemistry of the heart with the suture will be performed with primary staining for fibrin, platelets (α_{IIb}β₃ staining), vWF and thrombin.

Imaging of Prothrombotic Endothelial Phenotype in atherosclerosis

Imaging will be performed in 18-20 week old DKO mice that have a homozygous deletion of both the LDL receptor and the ApoBec editing enzyme that converts murine ApoB100 to ApoB48; or of control wild-type C57B1/6 mice. The DKO mice are characterized by aggressive atherosclerotic lesion development that is age-dependent and can result in lesion microthrombosis. Targeted CEU will be performed for the aortic arch 10 minutes after targeted or control microbubbles. Correlation between lesion development and targeted CEU signal will be made using high (40 MHz) imaging of the aortic arch and Masson's staining on pathology. Immunohistochemistry will be performed for vWF, thrombin, VCAM-1, α_{IIb}≈₃ (platelets), and tissue factor.

### Data Coordination and Analysis

During flow chamber studies, co-administration of differentially labeled control and GPIb-microbubbles will allow paired comparison. Likewise, paired analysis will be possible by co-administration for intravital microscopy experiments. Data for both will be stratified according to shear rates. Appropriate control data will be provided by evaluation of flow chambers without vWF or thrombin, or in microvessels without microvascular puncture. Imaging data for ventricular thrombus will be performed in a paired analysis (two-sided) using pre- and post-administration video intensity for both targeted and control microbubbles. Negative control threads are not possible in these situations due to the potential thrombogenicity of any foreign object in the vascular compartment. Video intensities of the ascending aorta and proximal aortic arch in atherosclerotic (DKO) control non-atherosclerotic mice will be compared for both targeted and non-targeted agents. For all experiments, the order of injection will be randomized.

### In Vivo CEU Imaging Studies

Spatial localization of VWF expression in vivo can be assessed by targeted CEU imaging of GPIb-microbubbles such as by injecting GPIb-microbubbles into mice that are deficient in ADAMTS-13, which is a physiologically relevant animal model of TTP (Chauhan et al. (2006) J. Exp. Med., 203:767-776; Motto et al. (2005) J. Clin. Invest., 115:2752-2761).

Attachment of VWF-targeted or control microbubbles in the microcirculation may be evaluated by intravital microscopy. The mesentery of anesthetized mice may be exteriorized and secured to a custom microscopy pedestal during isothermic buffered superfusion (Lindner et al. (2000) Circulation 102:531-538; Lindner et al. (2000) Circulation 102:2745-2750). Intravital microscopy (Axioskop2-FS, Carl Zeiss Inc.) of the microcirculation may be performed as previously described (Lindner et al. (2000) Circulation 101:668-675; Lindner et al. (2000) Circulation 102:531-538; Lindner et al. (2000) Circulation 102:2745-2750). Briefly, a 30-50 μm venule may be filmed for 3 minutes to establish the baseline before superfusion with calcium ionophore A23187 (a secretagogue of Weibel-Palade bodies) to induce VWF secretion. DiI-labeled GPIb- or DiO-labeled control microbubbles may be injected intravenously (5×10⁷ each). Fluorescently labeled, purified platelets (calcein AM) may be infused into the tail vein. The number of microbubbles and platelets attached may be determined by dual-fluorescent epiillumination. Flow and shear rates for the vascular segment may be determined from data on vessel diameter, calibrated with videocalipers and centerline velocity determined with a dual-slit photodiode.

For targeted CEU, a novel imaging protocol has been developed to detect signals from only retained microbubbles (Lindner, J. R. (2004) Nat. Rev. Drug Discov., 3:527-532; Lindner et al. (2000) Circulation 101:668-675; Lindner et al. (2000) Circulation 102:531-538; Lindner et al. (2000) Circulation 102:2745-2750). VWF-targeted or control microbubbles (1×10⁶) may be injected into the mouse. The dose may produce optimal signal to noise ratio in targeted tissues whereas signal intensity is essentially at the noise floor in normal tissues. CEU may be performed on the aortic arch in the long axis; a high right anterior thoracic approach may be used with the acoustic focus placed at the level of the arch (1 cm). Baseline grey-scale images may be acquired using broad-band (5-12 MHz) fundamental imaging. After superfusion of the calcium ionophore A23187 and after injection of VWF-targeted or control microbubbles, targeted CEU may be performed using a multipulse, harmonic Doppler (Angio) mode 10 minutes. A pulse interval of 20 seconds may be used for imaging, followed by an increase in pulse interval to 1 second to destroy the microbubbles. VWF-exposure may be correlated with targeted CEU signal using (40 MHz) imaging of the aortic arch and Masson's staining for the pathology. Immunohistochemistry of the endothelial surface may be performed with primary staining for VWF, fibrin, and platelets (α_{IIb}β₃ staining).

A paired analysis may performed by co-administration of microbubbles for intravital microscopy experiments. Data may be stratified according to shear rates. Appropriate control data may be provided by evaluating in microvessels prior to treatment with A23187. Imaging data for ventricular thrombus may be compared in a paired analysis (two-sided) using pre- and post-administration CEU intensity for both targeted and control microbubbles. CEU-intensities of the ascending aorta and proximal aortic arch in ADAMTS-13^{+/+} mice may be compared for both VWF-targeted and control agents.

For all experiments, the order of injection may be randomized.

### Studies

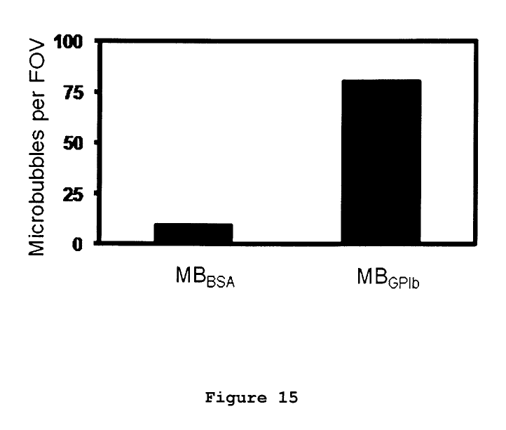

Suspensions of GPIb-labeled or BSA-labeled microbubbles (control) were drawn through a flow chamber coated with vWF. As seen in FIG. 15, microbubbles labeled with GPIb, but not BSA, attached to the vFW coated surface under a shear of 2 dyn/cm².

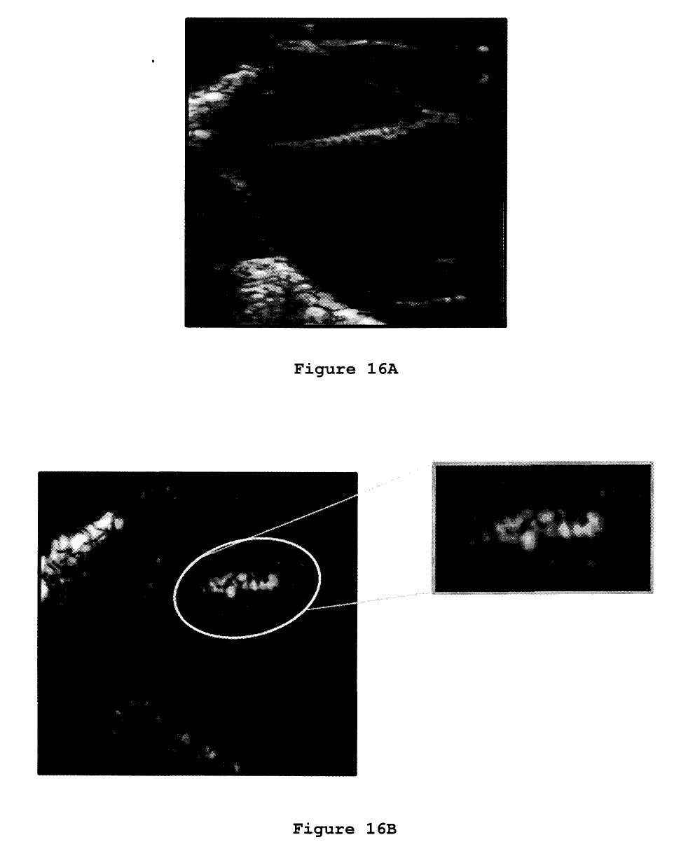

The ability of GPIb-labeled microbubbles to attach to a clot in vivo was also determined. As seen in FIG. 16, GPIbα conjugated microbubbles attached specifically to a clot in the left ventricle of a rat. The image was taken approximately 5 minutes after the injection, allowing the majority of the MB_{GPIb} bubbles to disperse.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. (canceled)

2. A method of detecting a cardiovascular disorder in a subject comprising:
a) administering microbubbles comprising glycoprotein Ib (GPIb) to said subject; and
b) determining the vascular retention of said microbubbles,
wherein an increase in the retention of microbubbles in said subject compared to the retention of microbubbles in a control subject who does not have a cardiovascular disorder indicates the presence of a cardiovascular disorder in said subject.

3. The method of claim 2, wherein step b) is performed by contrast-enhanced ultrasound.

4. The method of claim 2, wherein said cardiovascular disorder is selected from the group consisting of thrombosis, a prothrombotic environment, and atherosclerosis.

5. The method of claim 4, wherein said cardiovascular disorder is thrombotic thrombocytopenic purpura.

6. A method of detecting cardiovascular disease in a subject comprising:
a) administering microbubbles comprising a targeting ligand specific for VCAM-1 to said subject; and
b) determining the vascular retention of said microbubbles,
wherein an increase in the retention of microbubbles in said subject compared to the retention of microbubbles in a control subject who does not have cardiovascular disease indicates the presence of cardiovascular disease in said subject.

7. The method of claim 6, wherein step b) is performed by contrast-enhanced ultrasound.

8. The method of claim 6, wherein said targeting ligand specific for VCAM-1 is an antibody or antibody fragment.

9. The method of claim 6, wherein said cardiovascular disease is selected from the group consisting of atherosclerosis, myocardial injury, ischemia-mediated angiogenesis, and left ventricular ischemia.

10. A method of detecting a cardiovascular disorder in a subject comprising:
a) administering microbubbles comprising a targeting ligand specific for P-selectin to said subject; and
b) determining the vascular retention of said microbubbles,
wherein an increase in the retention of microbubbles in said subject compared to the retention of microbubbles in a control subject who does not have a cardiovascular disorder indicates the presence of a cardiovascular disorder in said subject.

11. The method of claim 10, wherein step b) is performed by contrast-enhanced ultrasound.

12. The method of claim 10, wherein said targeting ligand specific for P-selectin comprises PSGL-1 linked to a dimerization domain.

13. The method of claim 12, wherein said dimerization domain is an Fc domain.

14. The method of claim 10, wherein said cardiovascular disorder is selected from atherosclerosis, inflammation, and ischemia.

15. A composition comprising:
a) microbubbles comprising a targeting ligand, and
b) a carrier,
wherein said targeting ligand is selected from the group consisting of glycoprotein Ib (GPIb), a targeting ligand specific for VCAM-1, and a targeting ligand specific for P-selectin.

16. The composition of claim 15, wherein said targeting ligand specific for P-selectin comprises PSGL-1 linked to a dimerization domain.

17. The composition of claim 15, wherein said targeting ligand specific for VCAM-1 is an antibody or antibody fragment.
